(19) United States
(12) Patent Application Publication
(10) Pub. No.: US 2025/0064658 A1
(43) Pub. Date: Feb. 27, 2025

(54) COMMUNICATION SYSTEM FOR PATIENT SUPPORT APPARATUSES

(71) Applicant: Stryker Corporation, Kalamazoo, MI (US)

(72) Inventors: Madhu Sandeep Thota, Portage, MI (US); Anish Paul, Portage, MI (US); William Dwight Childs, Plainwell, MI (US); Krishna Sandeep Bhimavarapu, Kalamazoo, MI (US); Kirby M. Neihouser, Portage, MI (US); Celso Henrique Farnese Pires Pereira, Portage, MI (US); Madhu Thomas, London, Ontario (CA); Jerald A. Trepanier, Augusta, MI (US); Ramsey H. Othman, Portage, MI (US)

(21) Appl. No.: 18/724,899

(22) PCT Filed: Feb. 2, 2023
(86) PCT No.: PCT/US2023/012173

§ 371 Date: Jun. 27, 2024

Related U.S. Application Data
(60) Provisional application No. 63/306,279, filed on Feb. 3, 2022.
(60) Provisional application No. 63/426,450, filed on Nov. 18, 2022.
(60) Provisional application No. 63/428,074, filed on Nov. 27, 2022.

(51) Int. Cl. A61G 7/05; G16H 40/63

(52) U.S. Cl. CPC A61G 7/05; G16H 40/63; A61G 2203/30

(57) ABSTRACT

A patient support apparatus for supporting a patient includes first, second, and third transceivers mounted at first, second, and third locations, respectively. A controller is adapted to use radio frequency communication between the first, second, and third transceivers and a first device—such as a display device, a fixed locator, and/or a vital sign sensor—to determine a position of the first device relative to the patient support apparatus. The controller is further adapted to send data to a display device if the first device is positioned inside a predetermined volume of space, and to not send the data to the display device if the first device is positioned outside of the predetermined volume of space. The patient support apparatus may also selectively accept and communicate with a removable cartridge that includes a vital sign port adapted to receive a plug of a cable from a vital sign sensor.

### BACKGROUND

The present disclosure relates to patient support apparatuses, such as beds, cots, stretchers, recliners, or the like. More specifically, the present disclosure relates to patient support apparatuses that communicate with one or more devices that are separate from the patient support apparatus itself.

Patients in hospitals often have one or more of their vital signs monitored during their stay in the hospital. These vital sign readings are often recorded in an electronic medical record associated with a particular patient. In order for the vital sign readings from a particular vital sign sensor to be recorded in the correct patient's electronic medical record, a caregiver typically has to take one or more manual steps to associate the vital sign sensor with a particular patient. In order for the caregiver to see the vital signs reading, the caregiver typically has to plug a cable from the vital sign sensor into a display device and/or take other steps to view the vital sign readings.

### SUMMARY

According to the various aspects described herein, the present disclosure is directed to a patient support apparatus that reduces the labor associated with reading, recording, and/or displaying vital sign data from a patient. In some aspects, the patient support apparatus automatically detects the relative position of a vital sign sensor to the patient support apparatus and automatically associates the data from that particular vital sign sensor to the patient assigned to that patient support apparatus if the relative position meets one or more criteria. Alternatively, or additionally, the patient support apparatus automatically detects the relative position of a display device to the patient support apparatus and sends data to the display device only if the relative position falls within a predetermined volume of space. Additionally, or alternatively, the patient support apparatus may automatically detect the relative position of a fixed locator to the patient support apparatus and send data to be displayed to the fixed locator only if the relative position falls within a predetermined volume of space. In still other aspects, the patient support apparatus may be constructed with a removable cartridge adapted to receive one or more plugs from one or more vital sign sensors. The removable cartridge is removable from the patient support apparatus, is able to communicate with the patient support apparatus when removed therefrom, and is adapted to simplify cable management by allowing cable(s) from one or more vital sign sensors to remain plugged into the cartridge while the cartridge is removed from the patient support apparatus. The cartridge allows the patient to exit the patient support apparatus without having to unplug any cables running between a vital sign sensor and a display device adapted to display readings from the vital sign sensor. Still other improvements and/or advantages over prior art patient support apparatuses and communication systems will become apparent in light of the following written description and the accompanying drawings.

According to a first aspect of the present disclosure, a patient support apparatus is provided that includes a support surface; a first transceiver coupled to a first location on the patient support apparatus; a second transceiver coupled to a second location on the patient support apparatus; a third transceiver coupled to a third location on the patient support apparatus; and a controller. The controller is adapted to use radio frequency (RF) communication between the first, second, and third transceivers and a fixed locator positioned off-board the patient support apparatus to determine a position of the fixed locator relative to the patient support apparatus. The controller is further adapted to send data to be displayed by a display device positioned off-board the patient support apparatus if the fixed locator is positioned inside a predetermined volume of space, and to not send the data to be displayed by the display device if the fixed locator is positioned outside of the predetermined volume of space.

According to another aspect of the present disclosure, a patient support apparatus is provided that includes a support surface, a first transceiver, a second transceiver, a third transceiver, and a controller. The first transceiver is coupled to a first location on the patient support apparatus. The second transceiver is coupled to a second location on the patient support apparatus. The third transceiver is coupled to a third location on the patient support apparatus. The controller is adapted to use radio frequency (RF) communication between the first, second, and third transceivers and a display device to determine a position of the display device relative to the patient support apparatus. The display device is positioned off-board the patient support apparatus and includes a display. The controller is further adapted to send data to the display device to be displayed thereon if the display device is positioned inside a predetermined volume of space, and to not send the data to the display device if the display device is positioned outside of the predetermined volume of space.

According to another aspect of the present disclosure, a patient support apparatus is provided that includes a support surface, a first transceiver, a second transceiver, a third transceiver, and a controller. The support surface is adapted to support a patient thereon. The first transceiver is coupled to a first location on the patient support apparatus. The second transceiver is coupled to a second location on the patient support apparatus. The third transceiver is coupled to a third location on the patient support apparatus. The controller is adapted to use radio frequency (RF) communication between the first, second, and third transceivers and a vital sign sensor to determine a position of the vital sign sensor relative to the patient support apparatus. The controller is further adapted to receive vital sign data from the vital sign sensor, to send the vital sign data to a display device if the vital sign sensor is positioned inside a predetermined volume of space, and to not send the vital sign data to the display device if the vital sign sensor is positioned outside of the predetermined volume of space.

A patient support apparatus according to still another aspect of the present disclosure includes a support surface, a cartridge, a cartridge port, a cartridge receiver, and a controller. The support surface is adapted to support a patient thereon. The cartridge is adapted to be physically attached to, and removed from, the patient support apparatus. The cartridge includes a vital sign port adapted to receive a plug of a cable from a vital sign sensor adapted to detect a vital sign of the patient. The cartridge further includes a cartridge transmitter adapted to transmit a cartridge ID and vital sign data to the patient support apparatus. The cartridge port is adapted to physically receive the cartridge when the cartridge is physically attached to the patient support apparatus. The cartridge receiver adapted to communicate with the cartridge transmitter and to receive the cartridge ID and the vital sign data communicated from the cartridge transmitter. The controller is adapted to associate the cartridge with the patient support apparatus.

A patient support apparatus according to yet another aspect of the present disclosure includes a support surface, a cartridge, a cartridge port, a cartridge receiver, and a controller. The support surface is adapted to support a patient thereon. The cartridge is adapted to be physically attached to, and removed from, the patient support apparatus. The cartridge includes a vital sign port adapted to receive a plug of a cable from a vital sign sensor adapted to detect a vital sign of the patient. The cartridge further includes a cartridge transmitter adapted to transmit vital sign data to the patient support apparatus. The cartridge port is adapted to physically receive the cartridge when the cartridge is physically attached to the patient support apparatus. The cartridge receiver is adapted to communicate with the cartridge transmitter and to receive the vital sign data from the cartridge transmitter. The controller is adapted to send the vital sign data to a display device.

A patient support apparatus according to still another aspect of the present disclosure includes a support surface adapted to support a patient, a first transceiver coupled to a first location on the patient support apparatus, and a controller adapted to use radio frequency (RF) communication between the first transceiver and a fixed locator positioned off-board the patient support apparatus to determine a position of the fixed locator relative to the patient support apparatus. The controller is further adapted to send data to be displayed to a display device positioned off-board the patient support apparatus if at least one of the following is true: (a) the fixed locator is positioned inside a first predetermined volume of space defined with respect to the patient support apparatus; or (b) the patient support apparatus is positioned inside of a second predetermined volume of space defined with respect to the fixed locator. The controller is also configured to not send the data to be displayed to the display device if at least one of the following is true: (i) the fixed locator is positioned outside of the first predetermined volume of space, or (ii) the patient support apparatus is positioned outside of the second predetermined volume of space.

A patient support apparatus according to still another aspect of the present disclosure includes a support surface adapted to support a patient, a first transceiver coupled to a first location on the patient support apparatus, and a controller adapted to use radio frequency (RF) communication between the first transceiver and a display device positioned off-board the patient support apparatus to determine a position of the display device relative to the patient support apparatus. The controller is further adapted to send data to be displayed to the display device positioned off-board the patient support apparatus if the display device is positioned inside a predetermined volume of space defined with respect to the patient support apparatus. The controller still further adapted to not send the data to be displayed to the display device if the display device is positioned outside of the predetermined volume of space.

According to other aspects of the present disclosure, the first transceiver, the second transceiver, and the third transceiver may all be ultra-wideband transceivers.

In some aspects, the patient support apparatus further includes a memory in which the first location, second location, and third location of the first, second, and third transceivers, respectively, is stored. In such embodiments, the controller is further adapted to use the stored locations of the first, second, and third transceivers to determine the relative position of the fixed locator, the vital sign sensor, and/or the display device relative to the patient support apparatus. The controller is further adapted to use the determination of the relative position of the fixed locator, vital sign sensor, and/or display device to determine if the fixed locator, vital sign sensor, and/or display device is/are positioned inside or outside of the predetermined volume of space.

In some aspects, the controller is further adapted to include readings from the vital sign sensor within the data to be displayed by the display device if the vital sign sensor is positioned inside the predetermined volume of space, and to not include the readings from the vital sign sensor within the data to be displayed by the display device if the vital sign sensor is positioned outside of the predetermined volume of space.

In some aspects, the display device is mounted adjacent a bay of a room within a healthcare facility.

The patient support apparatus, in some aspects, further includes a microphone adapted to convert sounds of the patient's voice to audio signals, and the controller is further adapted to transmit the audio signals to the fixed locator if the fixed locator is positioned inside the predetermined volume of space, and to not transmit the audio signals to the fixed locator if the fixed locator is positioned outside of the predetermined volume of space.

In some aspects, the display device includes a device controller and a video port adapted to receive a video cable adapted to be coupled to a display. The device controller is adapted, in response to receiving the data to be displayed, to send the data to be displayed to the video port.

The video port, according to some aspects, includes one of a High-Definition Multimedia Interface (HDMI) connector, a Video Graphics Array (VGA) connector, a DisplayPort (DP) connector, a plurality of Radio Corporation of America (RCA) connectors, or a Digital Visual Interface (DVI) connector.

The patient support apparatus, according to some aspects, includes a vital sign port adapted to receive a plug of a cable from a vital sign sensor adapted to detect a vital sign of the patient. The controller is further adapted to include readings from the vital sign sensor within the data to be displayed by the display device.

The vital sign port, in some aspects, is integrated into a cartridge adapted to be removed from the patient support apparatus while the plug of the cable from the vital sign sensor remains plugged into the vital sign port.

The cartridge, in some aspect, includes an adapter and the cartridge is adapted to be removed from the patient support apparatus with the adapter connected to the cartridge. The adapter is adapted to be removed from the cartridge while the plug of the cable remains coupled to the adapter.

The cartridge, in some aspects, is removably secured to the patient support apparatus via an engineering fit, and the adapter is removably secured to the cartridge by magnetic coupling.

In some aspects, the cartridge further includes a battery and the cartridge transmitter is adapted to wirelessly transmit the vital sign data to the cartridge receiver when the cartridge is removed from the patient support apparatus.

The controller is adapted, in some aspects of the present disclosure, to include a room number within the data to be displayed by the display device. The room number identifies the room in which the patient support apparatus is currently located.

The patient support apparatus, in some aspects, further includes a network transceiver adapted to communicate with a server of a local area network of a healthcare facility, and the controller is adapted to receive a location ID from the fixed locator, to send the location ID to the server using the network transceiver, and to thereafter receive the room number from the server via the network transceiver.

In some aspects, the fixed locator is adapted to be mounted at a fixed location within a room of a healthcare facility and to transmit a location ID to the patient support apparatus.

The patient support apparatus, in some aspects, further includes a local display integrated therein and the controller is further adapted to display a screen on the local display that includes the data to be displayed by the display device.

The data to be displayed by the display device, in some aspects, includes readings from the vital sign sensor.

In some aspects, the predetermined volume of space is defined with respect to the patient support apparatus and moves as the patient support apparatus moves.

The patient support apparatus, in some aspects, further include a user interface adapted to allow a user to control a content of the data to be displayed by the display device.

In some aspects, the display device, vital sign sensor, and/or fixed locator includes a fourth transceiver adapted to communicate with the first, second and third transceivers. The fourth transceiver may be an ultra-wideband transceiver.

The display device, in some aspects, is one of a smart phone, a tablet computer, or a laptop computer.

In some aspects, the controller is adapted to send the data to be displayed directly to the display device without first sending the data to be displayed to any intermediary devices positioned off-board the patient support apparatus.

The controller, in some aspects, is adapted to send the data to be displayed to the fixed locator, and the fixed locator is adapted to forward the data to the display device.

The controller, in some aspects, is further adapted to include readings from the vital sign sensor within the data to be displayed if the vital sign sensor is positioned inside the predetermined volume of space, and to not include the readings from the vital sign sensor within the data to be displayed if the vital sign sensor is positioned outside of the predetermined volume of space.

In some aspects, the controller is further adapted to send the vital sign data to the display device if the display device is positioned inside the predetermined volume of space, and to not send the vital sign data to the display device if the display device is positioned outside of the predetermined volume of space.

The controller, in some aspects, is further adapted to send a room number to the display device if the display device is positioned inside the predetermined volume of space, and to not send the room number to the display device if the display device is positioned outside of the predetermined volume of space.

The patient support apparatus, in some aspects, includes a user interface adapted to allow a user to control a format of the vital sign data sent to the display device.

The user interface, in some aspects, is adapted to allow a user to select what data is to be sent to the display device.

The patient support apparatus, in some aspects, further includes a patient ID reader adapted to wirelessly receive a patient ID from a patient ID device, and a network transceiver adapted to communicate with a server. The controller is further adapted to send both the vital sign data and patient ID to the server.

According to other aspects of the present disclosure, the patient support apparatus may further include a network transceiver adapted to communicate with a server hosted on a computer network, and the controller may be adapted to transmit the cartridge ID and a patient support apparatus ID to the server.

The cartridge transmitter, in some aspects, is further adapted to transmit the cartridge ID during an association period in which the controller associates the cartridge with the patient support apparatus, and to subsequently transmit the cartridge ID during a post-association period. The controller is further adapted to check to see if the cartridge ID received during the post-association period matches the cartridge ID received during the association period.

In some aspects, the controller is further adapted to perform at least one of the following: (a) display an error message on a display if the cartridge ID received during the post-association period does not match the cartridge ID received during the association period; or (b) stop displaying the vital sign data on the display if the cartridge ID received during the post-association period does not match the cartridge ID received during the association period.

In some aspects, the controller may be adapted to perform at least one of the following: (a) send an error message to a server if the cartridge ID received during the post-association period does not match the cartridge ID received during the association period; or (b) stop sending the vital sign data to the server if the cartridge ID received during the post-association period does not match the cartridge ID received during the association period.

The patient support apparatus, in some aspects, further includes a patient ID reader adapted to wirelessly receive a patient ID from a patient ID device.

The controller, in some aspects, is adapted to send both the vital sign data and the patient ID to the server.

The patient ID device, in some aspects, is a wristband worn by the patient and the wristband includes a code imprinted thereon adapted to be read by the patient ID reader. The code identifies the patient.

In some aspects, the display device and the fixed locator are mounted inside of a common room within a healthcare facility.

In some aspects, the fixed locator is mounted inside of a room of a healthcare facility and the display device is mounted outside of the room.

In some aspects, the display device is a stationary display device and the controller is further adapted to send the data to be displayed to a mobile display device if the mobile display device is positioned inside of the first predetermined volume of space, but to not send the data to be displayed to the mobile display device if the mobile display device is positioned outside of the first predetermined volume of space.

The controller, in some aspects, is adapted to wirelessly send the data to be displayed to the display device.

The controller, in some aspects, is adapted to receive an identifier for the display device from the fixed locator and to use the identifier when sending the data to be displayed to the display device.

In some aspects, the patient support apparatus includes a network transceiver adapted to communicate with a server on a computer network, and the controller is further adapted to receive an identifier for the display device from the server and to use the identifier when sending the data to be displayed to the display device.

In some aspects, the controller is adapted to use radio frequency (RF) communication between the first transceiver and a fixed locator positioned off-board the patient support apparatus to determine a position of the fixed locator relative to the patient support apparatus. The controller is further adapted to send the data to be displayed to a second display device positioned off-board the patient support apparatus if at least one of the following is true: (a) the fixed locator is positioned inside a second predetermined volume of space defined with respect to the patient support apparatus; or (b) the patient support apparatus is positioned inside of a third predetermined volume of space defined with respect to the fixed locator. The controller is further adapted to not send the data to be displayed to the second display device if at least one of the following is true: (i) the fixed locator is positioned outside of the second predetermined volume of space, or (ii) the patient support apparatus is positioned outside of the third predetermined volume of space.

In some aspects, the controller is adapted to receive an identifier for the second display device from the fixed locator and to use the identifier when sending the data to be displayed to the second display device.

In some aspects, the patient support apparatus further includes a network transceiver adapted to communicate with a server on a computer network, and the controller is further adapted to receive an identifier for the second display device from the server and to use the identifier when sending the data to be displayed to the second display device.

Before the various aspects of the disclosure are explained in detail, it is to be understood that the claims are not to be limited to the details of operation or to the details of construction and the arrangement of the components set forth in the following description or illustrated in the drawings. The aspects described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first aspect of the present disclosure;

FIG. 2 is a plan view of an illustrative caregiver control panel of the patient support apparatus of FIG. 1;

FIG. 3 is a plan view of an illustrative patient control panel of the patient support apparatus of FIG. 1;

FIG. 4 is a perspective view of the patient support apparatus, a plurality of vital sign sensors, a display device, and a first type of locator unit that is used for automatically detecting the location of a patient support apparatus;

FIG. 5 is a block diagram of the patient support apparatus, vital sign sensors, and display device of FIG. 4, as well as a healthcare facility network;

FIG. 6 is a perspective view of the patient support apparatus, a plurality of vital sign sensors, a display device, and a second type of locator unit that is used for automatically detecting the location of the patient support apparatus;

FIG. 7 is a block diagram of the patient support apparatus, vital sign sensors, display device, and locator unit of FIG. 6, as well as the healthcare facility network;

FIG. 8 is a diagram of the patient support apparatus, vital sign sensor, and display device according to a first operational arrangement;

FIG. 9 is a diagram of the patient support apparatus, vital sign sensor, and display device according to a second operational arrangement;

FIG. 10 is a diagram of the patient support apparatus, vital sign sensor, and display device according to a third operational arrangement;

FIG. 11 is a diagram of the patient support apparatus, vital sign sensor, locator unit, and display device according to a fourth operational arrangement;

FIG. 12 is a diagram of the patient support apparatus, vital sign sensor, locator unit, and display device according to a fifth operational arrangement;

FIG. 13 is a diagram of the patient support apparatus and display device according to a sixth operational arrangement;

FIG. 14 is a diagram of the patient support apparatus, locator unit, and display device according to a seventh operational arrangement;

FIG. 15 is a diagram of the patient support apparatus, locator unit, and display device according to an eighth operational arrangement;

FIG. 16 is a diagram of the patient support apparatus, locator unit, and display device according to a ninth operational arrangement;

FIG. 17 is an example of a display device showing an illustrative set of data that may be received from the vital sign sensor(s) and/or the patient support apparatus and displayed thereon;

FIG. 18 is an exploded perspective view of a cartridge that may be removably coupled to the patient support apparatus;

FIG. 19 is an exploded perspective view of the cartridge of FIG. 18 shown coupled to a plurality of vital sign sensors;

FIG. 20 is a perspective view of the patient support apparatus showing a first alternative location for the cartridge;

FIG. 21 is a perspective view of the patient support apparatus showing a second alternative location for the cartridge;

FIG. 22 is a perspective view of the patient support apparatus showing a third alternative location for the cartridge, as well as a plurality of vital sign sensors coupled to the cartridge;

FIG. 23 is a perspective view of the patient support apparatus showing a fourth alternative location for the cartridge;

FIG. 24 is a perspective view of the cartridge removed from the patient support apparatus and positioned within a vicinity of a display device;

FIG. 25 is a diagram of the patient support apparatus, locator unit, and a plurality of display devices according to a tenth operational arrangement;

FIG. 26 is a diagram of the patient support apparatus, locator unit, and a plurality of display devices according to an eleventh operational arrangement; and

FIG. 27 is a perspective view of an overhead boom and patient support apparatus according to another aspect of the present disclosure.

### DETAILED DESCRIPTION OF THE EMBODIMENTS

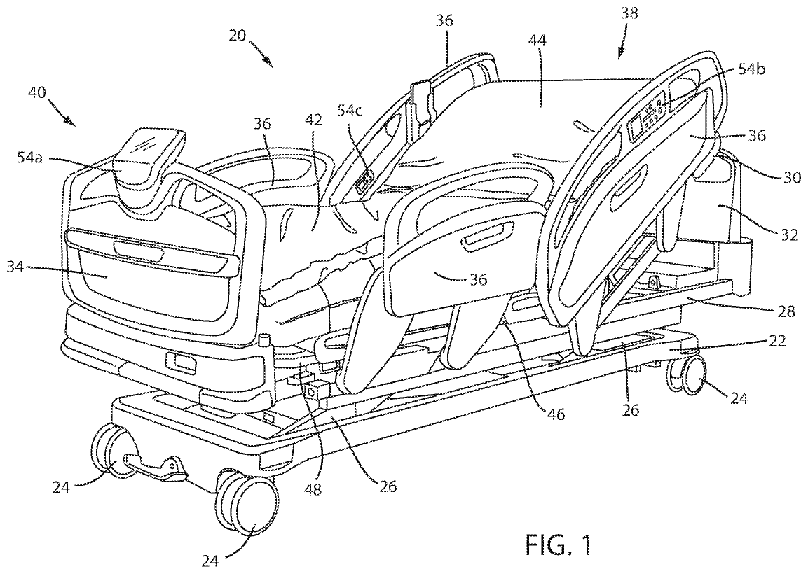

An illustrative patient support apparatus 20 according to an embodiment of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a recliner, an operating table, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon. In some embodiments, the mattress 42 includes one or more inflatable bladders that are controllable via a blower, or other source of pressurized air. In at least one embodiment, the inflation of the bladders of the mattress 42 is controllable via electronics built into patient support apparatus 20. In one such embodiments, mattress 42 may take on any of the functions and/or structures of any of the mattresses disclosed in commonly assigned U.S. Pat. No. 9,468,307 issued Oct. 18, 2016, to inventors Patrick Lafleche et al., the complete disclosure of which is incorporated herein by reference. Still other types of mattresses may be used.

Support deck 30 is made of a plurality of sections, some of which are pivotable about generally horizontal pivot axes. In the embodiment shown in FIG. 1, support deck 30 includes at least a head section 44, a thigh section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions that are different from what is shown in the attached drawings, such as, but not limited to, the construction described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may include the same, or nearly the same, structures as the Model 3002 S3 bed manufactured and sold by Stryker Corporation of Kalamazoo, Michigan. This construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. In still another embodiment, the mechanical construction of patient support apparatus 20 may include the same, or nearly the same, structure as the Model 3009 Procuity MedSurg bed manufactured and sold by Stryker Corporation of Kalamazoo, Michigan. This construction is described in greater detail in the Stryker Maintenance Manual for the 3009 Procuity MedSurg bed (publication 3009-009-002, Rev. A.0), published in 2020 by Stryker Corporation of Kalamazoo, Michigan.

It will be understood by those skilled in the art that patient support apparatus 20 can be designed with still other types of mechanical constructions, such as, but not limited to, those described in commonly assigned, U.S. Pat. No. 7,690,059 issued Apr. 6, 2010, to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The overall mechanical construction of patient support apparatus 20 may also take on still other forms different from what is disclosed in the aforementioned references provided the patient support apparatus includes one or more of the functions, features, and/or structures discussed in greater detail below.

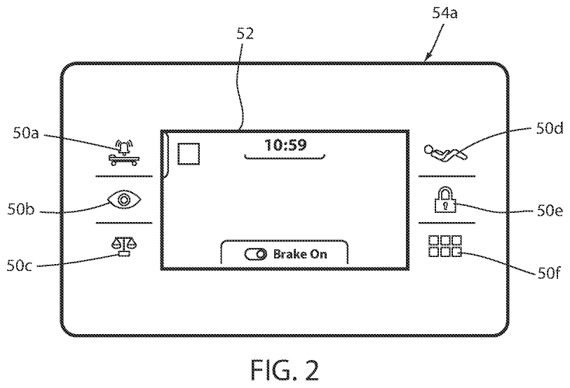

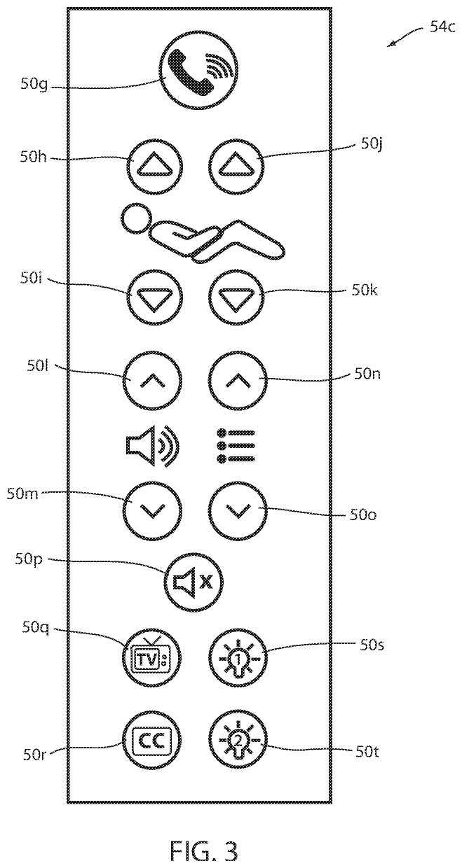

Patient support apparatus 20 further includes a plurality of control panels 54 that enable a user of patient support apparatus 20, such as a patient and/or an associated caregiver, to control one or more aspects of patient support apparatus 20. In the embodiment shown in FIG. 1, patient support apparatus 20 includes a footboard control panel 54a, a pair of outer siderail control panels 54b (only one of which is visible), and a pair of inner siderail control panels 54c (only one of which is visible). Footboard control panel 54a and outer siderail control panels 54b are intended to be used by caregivers, or other authorized personnel, while inner siderail control panels 54c are intended to be used by the patient associated with patient support apparatus 20. Each of the control panels 54 includes a plurality of controls 50 (see, e.g. FIGS. 2-3), although each control panel 54 does not necessarily include the same controls and/or functionality.

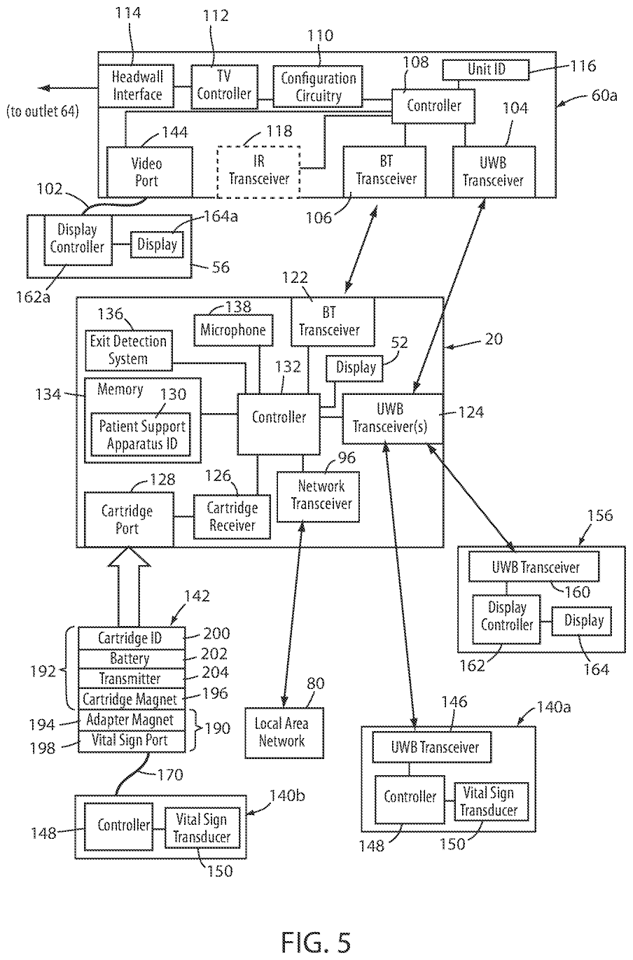

Among other functions, controls 50 of control panel 54a allow a user to control one or more of the following: change a height of support deck 30, raise or lower head section 44, activate and deactivate a brake for wheels 24, arm and disarm an exit detection system 136 (FIG. 5), change various settings on patient support apparatus 20, view the current location of the patient support apparatus 20 as determined by the location detection system discussed herein, view what vital sign sensors and/or other types of medical devices—if any—the patient support apparatus 20 has associated itself with, and perform other actions. One or both of the inner siderail control panels 54c also include at least one control that enables a patient to call a remotely located nurse (or other caregiver). In addition to the nurse call control, one or both of the inner siderail control panels 54c also include one or more controls for controlling one or more features of one or more room devices positioned within the same room as the patient support apparatus 20. As will be described in more detail below, such room devices include, but are not necessarily limited to, a television, a reading light, and a room light. With respect to the television, the features that may be controllable by one or more controls 50 on control panel 54c include, but are not limited to, the volume, the channel, the closed-captioning, and/or the power state of the television. With respect to the room and/or night lights, the features that may be controlled by one or more controls 50 on control panel 54c include the on/off state and/or the brightness level of these lights.

Control panel 54a includes a display 52 (FIG. 2) configured to display a plurality of different screens thereon. Surrounding display 52 are a plurality of navigation controls 50a-f that, when activated, cause the display 52 to display different screens on display 52. More specifically, when a user presses navigation control 50a, control panel 54a displays an exit detection control screen on display 52 that includes one or more icons that, when touched, control an onboard exit detection system 136 (FIG. 5). The exit detection system 136 is as adapted to issue an alert when a patient exits from patient support apparatus 20. Exit detection system 136 may include any of the same features and functions as, and/or may be constructed in any of the same manners as, the exit detection system disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. Other types of exit detection systems may be included within patient support apparatus 20.

When a user presses navigation control 50b (FIG. 2), control panel 54 displays a monitoring control screen that includes a plurality of control icons that, when touched, control an onboard monitoring system built into patient support apparatus 20. The onboard monitoring system alerts the caregiver through a unified indicator, such as a light or a plurality of lights controlled in a unified manner, when any one or more of a plurality of settings on patient support apparatus 20 are in an undesired state, and uses that same unified indicator to indicate when all of the plurality of settings are in their respective desired states. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20.

When a user presses navigation control 50c, control panel 54a displays a scale control screen that includes a plurality of control icons that, when touched, control the scale system of patient support apparatus 20. Such a scale system may include any of the same features and functions as, and/or may be constructed in any of the same manners as, the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system may utilize the same force sensors that are utilized by the exit detection system 136, or it may utilize one or more different sensors. Other scale systems besides those mentioned above in the '254 and '954 applications may alternatively be included within patient support apparatus 20.

When a user presses navigation control 50d, control panel 54 displays a motion control screen that includes a plurality of control icons that, when touched, control the movement of various components of patient support apparatus 20, such as, but not limited to, the height of litter frame 28 and the pivoting of head section 44. In some embodiments, the motion control screen displayed on display 52 in response to pressing control 50d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 50e, control panel 54a displays a motion lock control screen that includes a plurality of control icons that, when touched, control one or more motion lockout functions of patient support apparatus 20. Such motion lockout functions typically include the ability for a caregiver to use control panel 54a to lock out one or more of the motion controls 50 of the patient control panels 54c such that the patient is not able to use those controls 50 on control panels 54c to control the movement of one or more components of patient support apparatus 20. The motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

When a user presses on navigation control 50f, control panel 54a displays a menu screen that includes a plurality of menu icons that, when touched, bring up one or more additional screens for controlling and/or viewing one or more other aspects of patient support apparatus 20. Such other aspects include, but are not limited to, displaying information about one or more medical devices that are currently associated with patient support apparatus 20, diagnostic and/or service information for patient support apparatus 20, mattress control and/or status information, configuration settings, location information, and other settings and/or information. One example of a suitable menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20. In at least one embodiment, utilization of navigation control 50f allows a user to navigate to a screen that enables a user to see which medical devices, if any, are currently located within a predefined volume of space that encompasses patient support apparatus 20 and a predefined amount of space surrounding patient support apparatus 20. As will be discussed in greater detail below, patient support apparatus 20 includes an onboard locating system that is adapted to automatically determine the relative position of one or more medical devices with respect to patient support apparatus 20 and, in some instances, automatically associate those devices with patient support apparatus 20 (and/or the patient assigned to patient support apparatus 20) depending upon the proximity of the medical device to patient support apparatus 20. Further details of this locating system are provided below.

For all of the navigation controls 50a-f (FIG. 2), screens other than the ones specifically mentioned above may be displayed on display 52 in other embodiments of patient support apparatus 20 in response to a user pressing these controls. Thus, it will be understood that the specific screens mentioned above are merely representative of the types of screens that are displayable on display 52 in response to a user pressing on one or more of navigation controls 50a-f. It will also be understood that, although navigation controls 50a-f have all been illustrated in the accompanying drawings as dedicated controls that are positioned adjacent display 52, any one or more of these controls 50a-f could alternatively be touchscreen controls that are displayed at one or more locations on display 52. Still further, although controls 50a-f have been shown herein as buttons, it will be understood that any of controls 50a-f could also, or alternatively, be switches, dials, or other types of non-button controls. Additionally, patient support apparatus 20 may be modified to include additional, fewer, and/or different navigation controls from the navigation controls 50a-f shown in FIG. 2.

FIG. 3 illustrates one example of a patient control panel 54c that may be incorporated into patient support apparatus 20 and positioned at a location on patient support apparatus 20 that is convenient for a patient to access while supported on support deck 30, such as on an interior side of one of the siderails 36. Control panel 54c includes a plurality of controls 50g-t that are intended to be operated by a patient. A nurse call control 50g, when pressed by the patient, sends a signal to a nurse call system requesting that a remotely positioned nurse talk to the patient. A Fowler-up control 50h, when pressed by the patient, causes a motorized actuator onboard patient support apparatus 20 to raise Fowler section 44 upwardly. A Fowler-down control 50i, when pressed by the patient, causes the motorized actuator to lower Fowler section 44 downwardly. A gatch-up control 50j, when pressed by the patient, causes another motorized actuator to raise a knee section of support deck 30, while a gatch-down control 50k causes the motorized actuator to lower the knee section of support deck 30.

A volume-up control 50l, when pressed by the patient, causes patient support apparatus 20 to send a signal to an in-room television instructing it to increase its volume, while a volume down control 50m, when pressed, causes patient support apparatus 20 to send a signal to the television instructing it to decrease its volume. A channel-up control 50n, when pressed by the patient, causes patient support apparatus 20 to send a signal to the television instructing it to increase the channel number, while a channel-down control 50o, when pressed, causes patient support apparatus 20 to send a signal to the television instructing it to decrease the channel number.

A mute control 50p, when pressed, causes patient support apparatus 20 to send a signal to the television instructing it to either mute itself or unmute itself, depending upon whether the television is currently muted or unmuted. In other words, mute control 50p is a toggle control that alternatingly sends mute and unmute commands to the television when it is pressed.

Power control 50q is a toggle control that, when pressed, sends a signal to the television to either turn on or turn off, depending upon the television's current power status. Closed-captioning control 50r is another toggle control that, when pressed, sends a signal to the television to either turn on its closed-captioning feature or to turn off its closed captioning feature, depending upon whether the closed-captioning feature is currently on or off.

Control 50s is a toggle control that, when pressed, sends a signal to a first light to either turn on or turn off, depending upon the current state of that first light. Control 50t is another toggle control that, when pressed, sends a signal to a second light to either turn on or turn off, depending upon the current state of that second light. In some embodiments, the first light is a reading light and the second light is a room light, both of which are positioned off-board the patient support apparatus 20.

It will be understood that not only the number of controls 50 on control panel 54c, but also the functions of the controls 50 on control panel 54c, the layout of the controls 50 on control panel 54c, and/or other aspects of control panel 54c may be modified from what is shown in FIG. 3. In some embodiments, control panel 54c is implemented on a pendant controller that includes a cable that is plugged into a port on patient support apparatus 20. In other embodiments, one or more of the controls 50 of control panel 54c may be omitted, augmented, and/or split amongst other controls panels and/or locations. Still other manners of implementing control panel 54c are also possible.

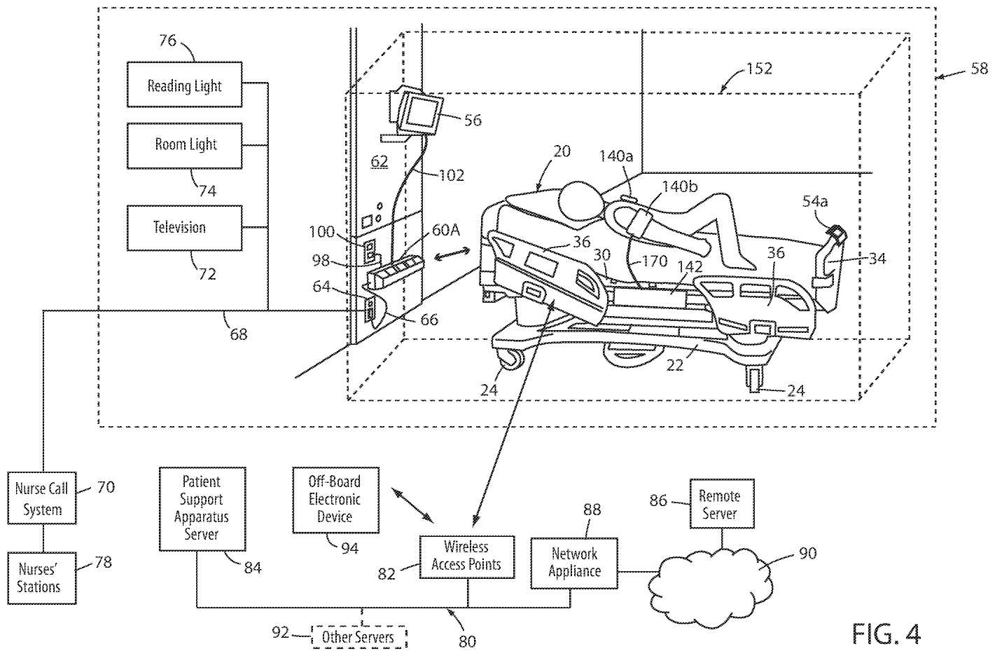

FIG. 4 illustrates patient support apparatus 20 positioned within a room 58 of a healthcare facility. FIG. 4 also illustrates the additional items that may be present in a healthcare facility and which patient support apparatus 20 is configured to communicate with, including, but not limited to, a locator unit 60 and a conventional local area network 80 of the healthcare facility. Locator units 60 are positioned at known and fixed locations within the healthcare facility in which patient support apparatus 20 is positioned. Locator units 60 function as fixed locators. That is, locator units 60 communicate with patient support apparatuses 20 and share information with them that allows the location of the patient support apparatuses 20 to be determined.

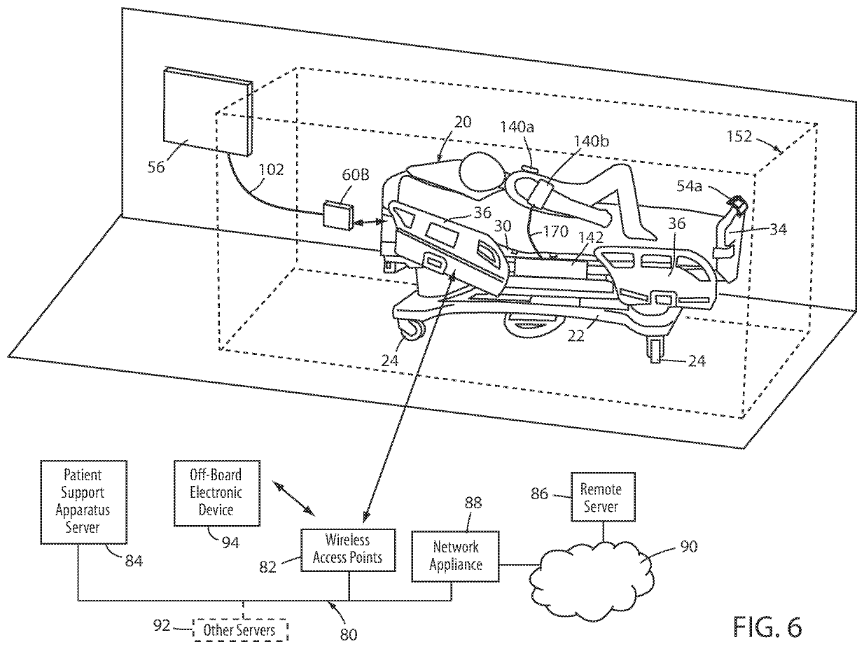

In some embodiments, patient support apparatus 20 is configured to be able to communicate with at least two different types of units: linked locator units 60a and unlinked locator units 60b. One example of a linked locator unit 60a is shown in FIG. 4. One example of an unlinked locator unit 60b is shown in FIG. 6. Locator units 60a and 60b differ from each other in that linked locator units 60a are adapted to communicate with a conventional communication outlet 64 that is typically built into one or more walls of a healthcare facility. That is, linked locator units 60a are communicatively linked to a conventional communication outlet 64. Unlinked locator units 60b are not adapted to communicate with such communication outlets 64, and are therefore not linked to a nearby communications outlet 64. Communications outlet 64 provides wired access to not only the healthcare facility's nurse call system 70, but also one or more room devices, such as a reading light 76, a room light 74, a television 72, and/or other devices.

Both locator units 60a and 60b are adapted to provide location information to patient support apparatus 20. Linked locator units 60a, however, are also adapted to serve as a communication conduit for routing communications between patient support apparatus 20 and one or more devices and/or systems that are communicatively coupled to communication outlet 64 (e.g. room devices 72, 74, 76, and/or nurse call system 70, FIG. 4). Unlinked locator units 60b, in contrast, are not adapted to serve as communication conduits between patient support apparatus 20 and these other devices and/or systems. In general, linked locator units 60a are typically positioned in patient rooms of the healthcare facility where one or more communication outlets 64 are typically present, while unlinked locator units 60b are typically positioned in locations outside of patient rooms, such as hallways, maintenance areas, and/or other areas. Unless explicitly stated otherwise, references herein to “locator units 60” refer to both locator units 60a and 60b. Both locator units 60a and 60b are adapted to enable the location of patient support apparatus 20 to be determined.

As shown in FIG. 4, linked locator units 60a are adapted to be mounted to a wall 62, such as a headwall of a patient room 58 within the healthcare facility. The headwall of a conventional healthcare facility room 58 typically includes a conventional communications outlet 64 physically integrated therein. Communications outlet 64 is adapted to receive a nurse call cable 66 that physically connects at its other end either to patient support apparatus 20 (not shown) or to linked locator unit 60a (shown in FIG. 4). In many healthcare facilities, communication outlet 64 includes a 37-pin connector, although other types of connectors are often found in certain healthcare facilities. As will be discussed in greater detail below, linked locator unit 60a and nurse call cable 66 allow patient support apparatus 20 to communicate with a nurse call system, and one or more room devices positioned within room 58.

Communication outlet 64 is electrically coupled to one or more cables, wires, or other conductors 68 that electrically couple the communication outlet 64 to a nurse call system 70 and one or more conventional room devices, such as a television 72, a room light 74, and/or a reading light 76. Conductors 68 are typically located behind wall 62 and not visible. In some healthcare facilities, conductors 68 may first couple to a room interface circuit board that includes one or more conductors 68 for electrically coupling the room interface circuit board to room device 72, 74, 76 and/or nurse call system 70. Still other communicative arrangements for coupling communication outlet 64 to nurse call system 70 and/or one or more room devices 72, 74, 76 are possible.

Nurse call cable 66 (FIG. 4) enables linked locator unit 60a to communicate with nurse call system 70 and/or room devices 72, 74, 76, and because patient support apparatus 20 is able to wirelessly communicate with linked locator unit 60a, patient support apparatus 20 is thereby able to communicate with nurse call system 70 and room devices 72, 74, 76. A patient supported on patient support apparatus 20 who activates a nurse call control (e.g. 50g; see FIG. 3) on patient support apparatus 20 causes a signal to be wirelessly sent from patient support apparatus 20 to linked locator unit 60a, which in turn conveys the signal via nurse call cable 66 to the nurse call system 70, which forwards the signal to one or more remotely located nurses (e.g. nurses at one or more nurse's stations 78). If the patient activates one or more room device controls (e.g. controls 50l-t; see FIG. 3), one or more wireless signals are conveyed to linked locator unit 60a, which in turn sends appropriate signals via nurse call cable 66 to communication outlet 64 and the room device 72, 74, 76 that change one or more features of these devices (e.g. the volume, channel, on/off state, etc.).

As is also shown in FIG. 4, patient support apparatus 20 is further configured to communicate with a local area network 80 of the healthcare facility. In the embodiment shown in FIG. 4, patient support apparatus 20 includes a wireless network transceiver 96 (FIG. 5) that communicates wirelessly with local area network 80. Network transceiver 96 is, in at least some embodiments, a WiFi transceiver (e.g. IEEE 802.11) that wirelessly communicates with one or more conventional wireless access points 82 of local area network 80. In other embodiments, network transceiver 96 may be a wireless transceiver that uses conventional 5G technology to communicate with network 80, one or more servers hosted thereon, and/or other devices. In some embodiments, network transceiver 96 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In some embodiments, network transceiver 96 is a wired transceiver that is adapted to allow patient support apparatus 20 to communicate with network 80 via a wired connection, such as an Ethernet cable that plugs into an Ethernet port (e.g. an RJ-45 style port, an 8P8C port, etc.) built into patient support apparatus 20. In still other embodiments, patient support apparatus 20 includes both a wired transceiver 96 for communicating with network 80 via a wired connection and a wireless transceiver 96 for wirelessly communicating with network 80.

Patient support apparatus 20 is configured to communicate with one or more servers on local area network 80 of the healthcare facility. One such server is a patient support apparatus server 84. Patient support apparatus server 84 is adapted, in at least one embodiment, to receive status information from patient support apparatuses 20 positioned within the healthcare facility and distribute this status information to caregivers, other servers, and/or other software applications. As will be discussed in greater detail below, server 84 may also be configured to receive data from one or more medical devices that are positioned within a volume of space defined around patient support apparatus 20 and/or within a volume of space defined around locator units 60. Alternatively, in some embodiments where data from medical devices is collected, the data from one or more of medical devices may be forwarded to one or more other servers 92 on network 80 (and/or one or more electronic devices 94), such as a caregiver assistance server and/or a caregiver assistance software application, as will also be discussed in greater detail below.

In some embodiments, patient support apparatus server 84 is configured to communicate at least some of the patient support apparatus status data and/or medical device data received from patient support apparatuses 20 to a remote server 86 that is positioned geographically remotely from the healthcare facility. Such communication may take place via a conventional network appliance 88, such as, but not limited to, a router and/or a gateway, that is coupled to the Internet 90. The remote server 86, in turn, is also coupled to the Internet 90, and patient support apparatus server 84 is provided with the URL and/or other information necessary to communicate with remote server 86 via the Internet connection between network 80 and server 86.

In some alternative embodiments, patient support apparatus 20 may be configured to communicate directly with one or more cloud-based servers, such as remote server 86, without utilizing patient support apparatus server 84. That is, in some embodiments, patient support apparatuses 20 may be configured to communicate directly with a remote server without relying upon any locally hosted servers (e.g. servers hosted on network 80). In one such embodiment, patient support apparatus 20 utilizes Microsoft's Azure could computing service to directly connect to one or more remote servers 86 without utilizing server 84. In some such embodiments, network appliance 88 is a router configured to support such direct connections. Still other types of direct-to-cloud connections may be utilized with one or more of patient support apparatuses 20.

Patient support apparatus server 84 is also configured to determine the location of each patient support apparatus 20, or receive the location of each patient support apparatus 20 from the patient support apparatuses 20. In some embodiments, patient support apparatus server 84 determines the room number and/or bay area of each patient support apparatus 20 that is positioned within a room 58, as well as the location of patient support apparatuses 20 that are positioned outside of a room 58, such as, those that may be positioned in a hallway, a maintenance area, or some other area. In general, patient support apparatus server 84 may be configured to determine the position of any patient support apparatus 20 that is positioned within communication range of one or more locator units 60, as will be discussed in greater detail below.

It will be understood that the architecture and content of local area network 80 will vary from healthcare facility to healthcare facility, and that the example shown in FIG. 4 is merely one example of the type of network a healthcare facility may be employ. Typically, one or more additional servers 92 will be hosted on network 80 and one or more of them may be adapted to communicate with patient support apparatus server 84. For example, an electronic health record server will typically be present in any healthcare facility, and in some embodiments discussed herein, it will be in communication with patient support apparatus server 84 in order to receive patient data that is to be recorded in a patient's health record (e.g. vital sign readings from one or more vital sign sensors; weight readings taken from the scales built into patient support apparatuses 20; therapies provided to patients using a powered mattress 42 onboard patient support apparatuses 20; data from other medical devices that are determined to be associated with the patient assigned to patient support apparatus 20, etc.). Local area network 80 will also typically allow one or more electronic devices 94 to access the local area network 80 via wireless access points 82. Such electronic devices 94 include, but are not limited to, smart phones, tablet computers, portable laptops, desktop computers, smart televisions, and other types of electronic devices that include a WiFi capability and that are provided with the proper credentials (e.g. SSID, password, etc.) to access network 80 (and, in at least some situations, patient support apparatus server 84).

Linked locator units 60a are adapted to wirelessly receive signals from patient support apparatus 20 and deliver the signals to communications outlet 64 in a manner that matches the way the signals would otherwise be delivered to communications outlet 64 if a conventional nurse call cable 66 were connected directly between patient support apparatus 20 and communications outlet 64. Linked locator units 60a are also adapted to transmit signals received from communications outlet 64 to patient support apparatus 20 via a BT transceiver 106 and/or a UWB transceiver 104 (FIG. 5). Thus, patient support apparatus 20 and linked locator unit 60a cooperate to send signals to, and receive signals from, communications outlet 64 in a manner that is transparent to communications outlet 64 such that outlet 64 cannot detect whether it is in communication with patient support apparatus 20 via a wired connection or it is in communication with patient support apparatus 20 via a wireless connection between patient support apparatus 20 and linked locator unit 60a (the latter of which is in wired communication with outlet 64). In this manner, a healthcare facility can utilize the wireless communication abilities of one or more patient support apparatuses 20 without having to make any changes to their existing communication outlets 64.

As noted, in addition to sending signals received from patient support apparatus 20 to communications outlet 64, linked locator units 60a are also adapted to forward signals received from communications outlet 64 to patient support apparatus 20. Linked locator units 60a are therefore adapted to provide bidirectional communication between patient support apparatus 20 and communications outlet 64. This bidirectional communication includes, but is not limited to, communicating command signals from any of controls 50 and/or from any of electronic devices 94 to corresponding room devices 72, 74, and/or 76 and communicating audio signals between a person supported on patient support apparatus 20 and a caregiver positioned remotely from patient support apparatus 20. The audio signals received by locator units 60 from a microphone on patient support apparatus 20 are forwarded to communications outlet 64 (for forwarding to nurse call system 70), and the audio signals of a remotely positioned nurse that are received at communications outlet 64 (from nurse call system 70) are forwarded to a speaker onboard patient support apparatus 20.

Nurse call cable 66, in some embodiments, includes a conventional 37 pin connector on each end, one of which is adapted to be inserted into outlet 64 and the other one of which is adapted to be inserted into locator unit 60. Such 37 pin connections are one of the most common types of connectors found on existing walls of medical facilities for making connections to the nurse call system 70 and room devices 72, 74, and 76. Linked locator unit 60a and nurse call cable 66 are therefore configured to mate with one of the most common type of communication outlets 64 used in medical facilities. Such 37 pin connectors, however, are not the only type of connectors, and it will be understood that linked locator units 60a can utilize different types of connectors that are adapted to electrically couple to different types of nurse call cables 66 and/or different types of communication outlets 64. One example of such an alternative communications outlet 64 and cable 66 is disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION, the complete disclosure of which is incorporated herein by reference. Still other types of communication outlets 64 and corresponding connectors may be utilized.

Linked locator unit 60a (FIG. 4) also includes an electrical cord 98 having a plug positioned at a far end that is adapted to be inserted into a conventional electrical outlet 100. Electrical cord 98 enables linked locator unit 60a to receive power from the mains electrical supply via outlet 100. It will be appreciated that, in some embodiments, linked locator unit 60a is battery operated and cord 98 may be omitted. In still other embodiments, linked locator unit 60a may be both battery operated and include cord 98 so that in the event of a power failure, battery power supplies power to linked locator unit 60a, and/or in the event of a battery failure, electrical power is received through outlet 100. Unlinked locator units 60b may also include a battery, electrical cord, or both.

In some embodiments, locator units 60 (60a and/or 60b) include a video port that is adapted to receive a display cable 102 (FIG. 4). The display cable 102 is adapted to couple to locator unit 60 at one end and a non-UWB display device 56 at its opposite send. Locator unit 60 is configured to use cable 102 send data to non-UWB display device 56 that is to be displayed thereon. Such data may include vital sign data from one or more vital sign sensors that are coupled to the patient on patient support apparatus 20, status data from one or more sensors onboard patient support apparatus 20, location data regarding the location of patient support apparatus 20, and/or other data. Some examples of the type of data that may be displayed on non-UWB display device 56 are shown in FIG. 16. Cable 102 may be a High-Definition Multimedia Interface (HDMI) cable, a Video Graphics Array (VGA) cable, a DisplayPort (DP) cable, a plurality of Radio Corporation of America (RCA) cables, a Digital Visual Interface (DVI) cable, and/or another type of cable. Locator unit 60 is configured to include a complementary type of connector that mates with a connector on an end of cable 102. Further details regarding the communication between patient support apparatus 20 and non-UWB display device 56 are provided below and, as will be discussed more herein, patient support apparatus 20 may be configured to communicate directly with certain display devices without using locator unit 60 as a communication intermediary.

In addition to any of the structures and functions described herein, locator units 60a (and 60b) are configured to communicate location data to patient support apparatus 20 that enables patient support apparatus 20 and/or patient support apparatus server 84 to determine the location of patient support apparatus 20 within the healthcare facility. In general, such location determination is carried out by patient support apparatus 20 analyzing wireless signals communicated between itself and locator unit 60 to its position relative to locator unit 60. After determining its relative position to locator unit 60, patient support apparatus 20 is configured to be able to have its absolute position within the healthcare facility determined by receiving a unique wall identifier (ID) from the locator unit 60. The location of each locator unit 60 in the healthcare facility is surveyed during the installation of locator units 60, and unique IDs of each locator unit 60 are also recorded during the installation of locator units 60. This surveying information and corresponding ID information may be stored in patient support apparatus server 84 and/or onboard patient support apparatus 20, thereby enabling patient support apparatus 20 and/or patient support apparatus server 84 to determine the location of a patient support apparatus 20 once its relative position to an identified locator unit 60 is known.

If the location of patient support apparatus 20 is determined remotely, patient support apparatus 20 sends its relative position information and the ID of the locator unit 60 (and its own unique patient support apparatus ID 130 (FIGS. 5 & 7)) to server 84. Server 84 includes a table of all of the locations of the locator units 60 (which, as noted, is generated via a surveying operation during the installation of locator units 60), and it uses that table to correlate the patient support apparatus IDs 130 and the locator unit IDs it receives to specific locations within the healthcare facility. Thus, if a particular patient support apparatus 20 (with a particular ID 130) sends a locator unit ID that corresponds to room 430, server 84 determines that that particular patient support apparatus 20 is currently located in room 430. Generally speaking, and as will be discussed in greater detail below, the location of a patient support apparatus 20 is deemed to correspond to whichever locator unit 60 it is currently associated with, and if it is not currently associated with any locator unit 60, its location may be indeterminate.

In some embodiments of patient support apparatus 20 and locator unit 60, the relative location of patient support apparatus 20 to a locator unit 60 is carried out solely using ultra-wideband communication between the patient support apparatus 20 and the locator unit 60. Alternatively, in some embodiments, patient support apparatus 20 solely uses short range infrared communications with locator unit 60 to determine its relative location, wherein such short range infrared communications are only possible when the patient support apparatus 20 is positioned within a close proximity to the locator unit 60 (e.g. in the range of about 1-3 meters). In these latter embodiments, patient support apparatus 20 may report that its location coincides with that of the nearby locator unit 60 when it is able to successfully communicate with the nearby locator unit 60 using these short range infrared communications. Still further, in some embodiments, patient support apparatus 20 and locator units 60 may communicate with each other using both infrared and ultra-wideband communications. Further details regarding the use of short range infrared communications for location determination are described in commonly assigned U.S. Pat. No. 9,999,375 issued Jun. 19, 2018, to inventors Michael Hayes et al. and entitled LOCATION DETECTION SYSTEMS AND METHODS, the complete disclosure of which is incorporated herein by reference.

In some embodiments, locator units 60 and/or patient support apparatuses 20 may be constructed to include any or all of the functionality of the wireless headwall units and/or patient support apparatuses disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION; in commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION; and/or in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosures of all of which are incorporated herein by reference.

Still further, in some embodiments, locator units 60 and/or patient support apparatuses 20 may be constructed to include any of the features and/or functions of the headwall units 144a and/or patient support apparatuses disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference.

FIG. 5 depicts a block diagram of patient support apparatus 20, a linked locator unit 60a, a plurality of vital sign sensors 140, a non-UWB display device 56, a UWB display device 156, network 80, and a cartridge 142 that is selectively removable from patient support apparatus 20. As will be discussed in greater detail below, patient support apparatus 20 is configured to automatically determine the location of one or more of the following: vital sign sensors 140, a UWB display device 156, a cartridge 142, and/or a linked locator unit 60a (or unlinked locator unit 60b) relative to patient support apparatus 20 and carry out communications with those devices if they are positioned within a defined proximity to patient support apparatus 20. In some embodiments, if the device is positioned within the defined proximity, patient support apparatus 20 automatically associates the device with the patient assigned to patient support apparatus 20, and causes data from that device (or devices) to be displayed on one or more of display devices 56 and/or 156, and/or forwards data from that device (or devices) to patient support apparatus server 84 and/or an electronic medical records (EMR) server in communication with network 80.

Linked locator unit 60a includes an ultra-wideband transceiver 104, a Bluetooth transceiver 106, a locator unit controller 108, configuration circuitry 110, a television controller 112, a headwall interface 114, a video port 144, a unit ID 116, and, in some embodiments, an infrared transceiver 118. Bluetooth transceiver 106 is adapted to communicate with a Bluetooth transceiver 122 onboard patient support apparatus 20 using RF waves in accordance with the conventional Bluetooth standard (e.g. IEEE 802.14.1 and/or the standard maintained by the Bluetooth Special Interest Group (SIG) of Kirkland, Washington, USA). In some embodiments, transceivers 106 and 122 utilize Bluetooth Low Energy communications.

Ultra-wideband transceiver 104 is adapted to communicate with one or more ultra-wideband transceivers 124 positioned onboard patient support apparatus 20. Transceiver 104 is adapted to determine a distance between itself and patient support apparatus 20. Alternatively, or additionally, transceiver 104 may be adapted to allow each transceiver 124 onboard patient support apparatus 20 to determine its distance from transceiver 104. In some embodiments, transceivers 104 and 124 use time of flight (TOF) computations to determine these distances. In other embodiments, transceivers 104 and 124 may utilize other techniques for determining their distances from each other, either in addition to, or in lieu of, TOF computations. In some embodiments, transceivers 104, 124 may also determine an angle between themselves using angular information derived from antenna arrays positions onboard transceivers 104, 124, or by using other techniques. The position and orientation of each transceiver 124 onboard patient support apparatus 20 is known and stored in an onboard memory and used to determine the position and orientation of patient support apparatus 20 with respect to the locator unit(s) 60 with which it is communicating.

In some embodiments, transceivers 104, 124 are implemented as any of the Trimension™ ultra-wideband modules available from NXP Semiconductors of Austin, Texas. These modules include, but are not limited to, the Trimension™ UWB modules ASMOP1BO0N1, ASMOP1CO0R1, and/or the ASMOP1CO0A1, that utilize any of the following chips: the NXP SR150, SR100T, SR040, NCJ29D5, and/or the OL23DO chips. Modules manufactured and/or marketed by other companies may also be used, including, but not limited to, the Decawave DWM1000, DWM10001C, DWM3000 modules (available from Decawave of Dublin, Ireland); the Nordic TSG5162 SiP module (available from Tsingoal Technology of Beijing, China); and/or the UWB hub, wand, and/or sensors available from Zebra technologies of Lincolnshire, Illinois. Still other types of UWB modules may be used to implement transceivers 104 and 124.

Locator unit controller 108 is adapted to control the operation of transceivers 104, 106, configuration circuitry 110, TV controller 112, headwall interface 114, video port 144, and, if included, IR transceiver 118 (FIG. 5). When infrared transceiver 118 is included, it may be included to provide backwards compatibility to patient support apparatuses 20 that are not equipped with a UWB transceiver 124. That is, some healthcare facilities may include one or more patient support apparatuses that are not equipped with a UWB transceiver 124, but that do include an IR transceiver that is adapted to communicate with IR transceiver 118. When linked locator unit 60a includes IR transceiver 118, it is able to communicate its unit ID 116 to such patient support apparatuses via IR transceiver 118, which is a short range transceiver that is configured to only communicate with an adjacent patient support apparatus when the patient support apparatus is nearby (e.g. without about five feet or so). Such an adjacent patient support apparatus 20 then communicates the received locator unit ID 116 along with its own unique ID 130 (FIGS. 5 & 7) to server 84 which, as noted previously, is able to correlate the locator unit ID 116 to a particular location with the healthcare facility. In this manner, server 84 is able to use linked locator units 60a determine the location of versions of patient support apparatuses 20 that don't have a UWB transceiver 104, but that do have an IR transceiver.

Headwall interface 114 is adapted to change the electrical state of one or more pins that are in electrical communication with communication outlet 64 (via cable 66). Headwall interface 114 changes these electrical states in response to instructions from controller 108. For example, if the exit detection system 136 of patient support apparatus 20 detects a patient exit, a controller 132 of patient support apparatus 20 sends an exit alert signal to linked locator unit 60a and controller 108 responds by instructing headwall interface 114 to change the electrical state of at least one pin that is used to signal an exit alert (or a generic priority alert) to the nurse call system 70 via communications outlet 64. In some embodiments, headwall interface 114 may be constructed in the same manner as, and/or may include any one or of the functions as, the cable interface 88 described in commonly assigned U.S. patent application Ser. No. 63/193,778 filed May 27, 2021, by inventors Krishna Bhimavarapu et al. and entitled PATIENT SUPPORT APPARATUS AND HEADWALL UNIT SYNCING, the complete disclosure of which is incorporated herein by reference. Alternatively, or additionally, headwall interface 114 may be constructed in the same manner as, and/or may include any one or more of the same functions as, the headwall interface 120 disclosed in commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which is incorporated herein by reference. Linked locator unit 60a may also be configured to perform any of the functions of the headwall units 94 disclosed in the above-mentioned '778 patent application.

Configuration circuitry 110 and TV controller 112 may be configured to perform any of the same functions as, and/or be constructed in any of the same manners as, the configuration circuitry 132 and the TV control circuit 134, respectively, of commonly assigned U.S. patent application Ser. No. 63/131,508 filed Dec. 29, 2020, by inventors Kirby Neihouser et al. and entitled TOOL FOR CONFIGURING HEADWALL UNITS USED FOR PATIENT SUPPORT APPARATUS COMMUNICATION, the complete disclosure of which has already been incorporated herein by reference. Additionally, or alternatively, linked locator unit 60a may be configured to perform any of the functions of the headwall units 144 disclosed in the aforementioned '508 patent application.

Patient support apparatus 20 includes a controller 132, a memory 134, exit detection system 136, a microphone 138, a cartridge receiver 126, a cartridge port 128, Bluetooth transceiver 122, one or more UWB transceivers 124, display 52, network transceiver 96, and a plurality of additional components that are not shown in FIG. 5. Each UWB transceiver 124 is positioned at a known location on patient support apparatus 20. This known location information is stored in memory 134 and/or elsewhere, and may be defined with respect to any suitable common frame of reference. The known location information may include the spatial relationship between UWB transceivers 124 and/or any other components of patient support apparatus 20. For example, in some embodiments, the known location information includes the spatial relationship not only between UWB transceivers 124, but also the spatial relationships between UWB transceivers 124 and one or more of the following: the head end 38 of patient support apparatus 20, the foot end of patient support apparatus 20, the sides of patient support apparatus 20, the floor, and/or other components and/or landmarks of patient support apparatus 20. In some embodiments, this location information is used to determine the orientation of patient support apparatus 20 with respect to one or more walls 62, locator units 60, another patient support apparatus 20, and/or another object or structure within the healthcare facility.

Controller 132, as well as controller 108, may take on a variety of different forms. In the illustrated embodiment, each of these controllers is implemented as a conventional microcontroller. However, these controllers may be modified to use a variety of other types of circuits-either alone or in combination with one or more microcontrollers-such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controllers 108 and 132 when carrying out the functions described herein, as well as the data necessary for carrying out these functions, are stored in a corresponding memory that is accessible to that particular controller (e.g. memory 134 for controller 132, and a memory (not shown) for controller 108). In some embodiments, controller 132 may include and/or work with a microcontroller that is integrated into, or associated with, UWB transceiver(s) 124, and controller 108 may include and/or work with a microcontroller that is integrated into, or associated with, UWB transceiver 104.

Controller 132 utilizes transceivers 124 to determine the relative position of patient support apparatus 20 with respect to one or more nearby locator units 60. If patient support apparatus 20 is positioned within range of a locator unit 60, its UWB transceiver 124 communicates with the UWB transceiver 104 positioned on that locator unit 60, and the two transceivers 124 and 104 exchange signals that enable them to determine the distance between themselves. The same is done for each UWB transceiver 124 positioned onboard patient support apparatus 20. In some embodiments, patient support apparatus 20 includes at least three UWB transceivers 124, and patient support apparatus 20 determines the distance between each one of these UWB transceiver 124 and UWB transceiver 104 of locator unit 60.

In some embodiments, UWB transceivers 104, 124 may also be configured to determine an angular relationship between themselves. The distance (and angle information) in at least some embodiments is calculated by UWB transceiver 124 and controller 132 of patient support apparatus 20. In other embodiments, UWB transceiver 104 and controller 108 may calculate the distance (and angle information) and forward the results of this calculation to patient support apparatus 20 (either via UWB transceiver 104 or BT transceiver 106). In either situation, patient support apparatus controller 132 is informed of the distances (and, in some embodiments, as noted, the angle information) between transceivers 124 and 104. These distances and orientations are then used to calculate a relative position of patient support apparatus 20 to the locator unit 60 in a common frame of reference that, as will be discussed in greater detail below, may be defined in a fixed relationship to the patient support apparatus 20 or in a fixed relationship to the locator unit 60.

Although FIG. 5 (and FIG. 6) only illustrate a single locator unit 60, it will be understood that a typical healthcare facility will include multiple locator units 60 positioned at different locations throughout the facility, including ones positioned within patient rooms and others positioned outside of patient rooms. Typically, at least one linked locator unit 60a will be positioned in each patient room of the healthcare facility, and if the patient room is intended to be occupied by more than one patient (e.g. it includes multiple bays), then additional linked locator units 60a may be included so that each patient support apparatus 20 will have a linked locator unit 60a positioned adjacent to each bay area in the room. Additional locator units 60, such as unlinked locator units 60b, may also be positioned at other locations through the healthcare facility.

The location of patient support apparatus 20 relative to locator units 60 is repetitively determined by an exchange of communication signals between UWB transceivers 104 and 124. This exchange is initiated by an interrogation signal that may be sent by the UWB transceivers 104 of the locator unit 60, and/or it may be sent by the UWB transceivers 124 of the patient support apparatuses 20. The trigger for sending these interrogation signals (from either source) may simply be the passage of a predefined interval of time, in at least some embodiments. That is, in some embodiments, patient support apparatus 20 and/or locator units 60 may be configured to periodically send out an interrogation signal that will be responded to by any UWB transceivers 104 or 124 that are positioned with range of that signal. In those embodiments where patient support apparatuses 20 are configured to send out such an interrogation signal, the time intervals between the interrogation signals may be varied depending upon the location and/or other status of the patient support apparatus 20. For example, in some embodiments, patient support apparatuses 20 may be configured to send out the interrogation signals with longer timer intervals between them when the patient support apparatus is stationary, and to send out the interrogation signals with shorter time intervals between them when the patient support apparatus 20 is in motion. Indeed, in some embodiments, after patient support apparatus 20 has ceased moving, controller 132 may be configured to cease sending out such interrogation signals until it once again starts moving. In any of the aforementioned embodiments, motion of the patient support apparatus 20 may be detected in any suitable manner, such as by including one or more motion sensors on the patient support apparatus 20 (e.g. one or more accelerometers), and/or by monitoring the values of the repetitive distance measurements and looking for changes indicative of movement.

The measured distances (and/or angular information between locator units 60 and patient support apparatuses 20) that are generated from the communications between UWB transceivers 104, 124 may utilize Angle of Arrival (AoA) information, Time of Flight (TOF) information, Channel State Information, Time Difference of Arrival (TDoA) information, Two-Way Ranging (TWR) ranging information, and/or other information. In some embodiments, each transceiver 104, 124 includes an array of antennas that are used to generate distance and/or angular information with respect to the transceiver 104, 124 in which it is in communication. Still further, in some embodiments, transceivers 104, 124 include one or more of their own microcontrollers, and the location of transceivers 104, 124 may be determined by these internal microcontrollers without utilizing controller 132 and/or 108. In other embodiments, controllers 108 and/or 132 may work in conjunction with the microcontrollers of transceivers 104, 124 to determine their relative locations to each other.

Patient support apparatus 20 also includes, in at least some embodiments, a microphone 138 (FIG. 5) that is used to detect the voice of the patient when the patient wants to speak to a remotely positioned nurse. The patient's voice is converted to audio signals by microphone 138 and controller 132 is adapted to forward these audio signals to an adjacent communications outlet 64 positioned in wall 62 (FIG. 4). When a cable 66 is coupled between patient support apparatus 20 and outlet 64, controller 132 forwards these audio signals to outlet 64 via the cable. When no such cable 66 extends between patient support apparatus 20 and outlet 64, controller 132 wirelessly forwards these audio signals to the locator unit 60 that it is currently associated with (using transceiver 122, or in some embodiments, transceiver 124)) and controller 108 of locator unit 60 forwards these audio signals to outlet 64. As was noted, outlet 64 is in electrical communication with a conventional nurse call system 70 that is adapted to route the audio signals to the correct nurse's station 78, and/or other location. In some embodiments, microphone 138 acts as both a microphone and a speaker. In other embodiments, a separate speaker may be included in order to communicate the voice signals received from the remotely positioned nurse. In some embodiments, the audio communication between patient support apparatus 20 and communications outlet 64 is carried out in any of the manners, and/or includes any of the structures, disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

After the installation of locator units 60a and 60b in a particular healthcare facility, the location of each locator unit 60 within that facility is recorded. In some embodiments, the coordinates of the locations of locator units 60 are recorded in a common frame of reference (or converted to a common frame of reference after recordation). Such coordinates may be three dimensional (i.e. include a height components), or they may be two dimensional (no height component). In other embodiments, a more generalized location of one or more locator units 60 is determined, rather than the precise coordinates of the locator units 60. In still other embodiments, the locations of one or more locator units 60 are determined both generally and more precisely. The generalized location of the locator units 60 may include an indication of the room, bay, area, hallway, portion of a hallway, wing, maintenance area, etc. that the locator unit 60 is positioned in. The specific location of the locator units 60, as noted, may include an X, Y, and Z coordinate within a common frame of reference.

Regardless of how the location of each locator unit 60 is initially determined after they are installed in a healthcare facility (e.g. whether their coordinates are determined or a more generalized location is determined), the locations of all of the locator units 60, as well as their unique IDs 116, are stored in a memory accessible to server 84. Server 84 then uses this location data and ID data to determine the location of a patient support apparatus 20. Alternatively, or additionally, the location data and ID data are forwarded to patient support apparatuses 20 for storage in their onboard memories and for use in determining their own locations. In some embodiments, the location of each locator unit 60 (whether specific and/or general) may also, or alternatively, be stored in a memory within that particular locator unit 60 and shared with the devices it communicates with (e.g. patient support apparatuses 20). In some other embodiments, the location of each locator unit 60 may be stored in multiple locations.

It will be appreciated that patient support apparatuses 20 are configured to communicate with locator units 60 regardless of the orientation of the patient support apparatus 20. That is, the UWB transceivers 104 and 124 are radio frequency transceivers that do not rely on line of sight communication, unlike the IR transceiver 118 (if present). Thus, the patient support apparatuses 20 do not have to be pointed in any particular direction with respect to the locator units in order for transceivers 104 and 124 to communicate. This differs from some prior art systems that use IR communication between the patient support apparatuses 20 and the locator units and that require the IR transceiver onboard the patient support apparatus to be aimed toward the locator unit in order for communication to be established.

It will also be understood that locator units 60 can be positioned on wall, columns, ceilings, or any other fixed structures within the healthcare facility.

Patient support apparatus 20 is also configured to use UWB transceivers 124 to determine the position of various other devices relative to patient support apparatus 20, such as one or more vital sign sensors 140, one or more UWB display devices 156, and/or a cartridge 142 that has been physically separated from patient support apparatuses 20 (FIG. 5). As will be discussed in greater detail below, controller 132 uses UWB transceivers 124 to determine the relative position of these devices by communicating with one or more respective UWB transceiver that are either built into those devices or attached to a tag that is affixed to those devices. Such UWB transceivers operate in the same manner as UWB transceivers 124 and/or UWB transceiver 104 of locator units 60. And, as will also be discussed in greater detail below, controller 132 of patient support apparatus 20 uses the relative position information to determine how it will interact with these devices, including whether to display data from these devices and/or whether to send data from one or more of these devices to patient support apparatus server 84 and/or an EMR server on network 80.

Vital sign sensor 140a includes a UWB transceiver 146, a controller 148, and one or more vital sign transducers 150. UWB transceiver 146 is adapted to communicate with the UWB transceivers 124 positioned onboard patient support apparatus 20 so that the position of vital sign sensor 140a relative to patient support apparatus 20 can be repetitively determined. UWB transceiver 146 may be the same as all of the other UWB transceivers discussed herein (e.g. UWB transceivers 124, 104, etc.). Controller 148 of vital sign sensor 140a is adapted to oversee the operation of vital sign sensor, process the communications of UWB transceiver 146 with other UWB transceivers (e.g. transceivers 124), and process the outputs of vital sign transducer 150. Vital sign transducer 150 may vary from vital sign sensor 140 to vital sign sensor 140. In some embodiments, where vital sign sensor 140 is adapted to detect a patient's blood pressure, vital sign transducer 150 may be a pressure sensor adapted to detect the pressure of the patient's blood. For other vital sign sensors 140, transducer 150 may be a thermometer for measuring the patient's temperature, a pressure or sound detector for measuring the patient's breathing rate, a sound detector for measuring the patient's heart rate, an infrared light detector for detecting reflected infrared light in a pulse oximeter, and/or any other type of transducer, sensor, or detector that is capable of detecting one or more vital signs, or other medically significant characteristics of the patient.

Controller 148 of vital sign sensor 140 may comprise a microcontroller and/or any of the other structures that were mentioned above and that comprise controller 132 and/or controller 108. Controller 148 is adapted to send vital signs readings derived from the transducer 150 to patient support apparatus 20 when the vital sign sensor 140 is associated with the patient support apparatus 20. As will be discussed in more detail below, controller 132 of patient support apparatus 20 decides whether to associate a particular device, such as a vital sign sensor 140, with patient support apparatus 20 (or the patient assigned to patient support apparatus 20) if it is currently positioned in a predefined location. In general, controller 132 associates devices, such as vital sign sensor 140, with patient support apparatus 20 if the device is positioned within a predetermined volume of space 152 (FIG. 4) that is defined around, and with respect to, patient support apparatus 20.

If controller 132 of patient support apparatus 20 concludes that a vital sign sensor 140 is currently positioned within predetermined space volume 152, and therefore associates that vital sign sensor 140 with patient support apparatus 20, controller 132 is configured to request and/or receive vital sign data from the vital sign sensor 140. Additionally, controller 132 is configured to forward that vital sign data to one or more recipients, such as a locator unit 60 (which may then forward it to a non-UWB display device 56 for display thereon), a UWB display device 156, one or more displays 52 positioned onboard patient support apparatus 20, and/or one or more servers on the local area network 80 of the healthcare facility. When forwarding the vital sign data from patient support apparatus 20 to a locator unit 60, controller 132 may utilize BT transceiver 122 to send the data to BT transceiver 106. When forwarding the vital sign data from patient support apparatus 20 to a server on network 80, controller 132 may utilize network transceiver 96 and one or more access points 82. And when forwarding the vital sign data from patient support apparatus 20 to a UWB display device 156, controller 132 may use one or more of its UWB transceivers 124 to transmit the vital sign data to a UWB transceiver that is part of the UWB display device 156.

As noted above, patient support apparatus 20 is also configured to use UWB transceivers 124 to determine the relative position of one or more UWB display devices 156 (FIG. 5). UWB display device 156 includes a UWB transceiver 160, a controller 162, and a display 164. UWB transceiver 160 is adapted to communicate with the UWB transceivers 124 positioned onboard patient support apparatus 20 so that the position of UWB display device 156 relative to patient support apparatus 20 can be repetitively determined. UWB transceiver 160 may be the same as all of the other UWB transceivers discussed herein (e.g. UWB transceivers 124, 104, 146, etc.). Controller 162 of UWB display device 156 is adapted to oversee the operation of UWB display device 156, process the communications of UWB transceiver 160 with other UWB transceivers, and to control what content is displayed on display 164. Display 164 may be a conventional LCD display, a cathode ray tube (CRT) tube display, a plasma screen display, and/or any other type of display that is capable of displaying the data discussed herein.

Controller 162 of UWB display device 156 (FIG. 5) may comprise a microcontroller and/or any of the other structures that were mentioned above and that comprise controller 132 and/or controller 108. Controller 162 is adapted to display vital signs readings and/or other data received from patient support apparatus 20 when controller 132 has determined that UWB display device 156 is associated with patient support apparatus 20 (e.g. positioned within predetermined space volume 152). Thus, for example, if controller 132 of patient support apparatus 20 associates both vital sign sensor 140a and UWB display device 156 with patient support apparatus 20, controller 132 is configured to receive the vital sign data from vital sign sensor 140a and, depending upon how a user has configured the settings on patient support apparatus 20, to send that vital sign sensor data to UWB display device 156. In this manner, for example, a vital sign sensor 140a that corresponds to a sphygmomanometer may send the patient's blood pressure readings to patient support apparatus 20 via transceiver 146, and controller 132 may forward those blood pressure readings to UWB display device 156 for displaying on display 164. Alternatively, or additionally, controller 132 may send those blood pressure readings to linked locator unit 60a for forwarding to non-UWB display device 56 via video port 144 and display cable 102. Alternatively, or in addition, controller 132 may forward the blood pressure readings to a server (e.g. server 84 and/or an EMR server) on network 80 via network transceiver 96.

As with vital sign sensors 140, controller 132 uses UWB transceivers 124 to determine the relative position of the UWB display device 156 by communicating with one or more respective UWB transceivers 160 that are either built into UWB display device 156 or attached to a tag that is affixed to UWB display device 156. Such UWB transceivers operate in the same manner as UWB transceivers 124 and/or UWB transceivers. The determination of the position of UWB display device 156 relative to patient support apparatus 20 is used by controller 132 to determine if it will associate UWB display device 156 with patient support apparatus 20, and thus whether or not it will send data to be displayed on UWB display device 156.

In some embodiments, patient support apparatus 20 is configurable by a user (e.g. via control panel 54a) to control what information, if any, patient support apparatus 20 will send to an associated display device 56 or 156. Thus, a user can instruct patient support apparatus 20, for example, to send blood pressure readings, breathing rate readings, and pulse rate readings to the UWB display device 156, but not patient temperature readings and/or other data. As another example, patient support apparatus 20 is configurable by a user such that controller 132 will not send any vital sign data to any display devices 56 or 156, even if controller 132 determines they (or their associated locator unit 60) are within the space volume 152 and associated them with patient support apparatus 20.

Locator units 60 are configured to send a message to patient support apparatuses 20 indicating whether or not a non-UWB display device 56 is coupled thereto via video port 144 (or, in some cases discussed in greater detail below, via a wireless connection). In such embodiments, when controller 132 of patient support apparatus 20 receives the signal indicating that a non-UWB display device 56 is coupled to an associated locator unit, it is configured to consult the customized user settings to determine whether to send vital sign data (or other data) to that locator unit 60 for forwarding to the non-UWB display device 56 coupled to video port 144. It bears noting that, when patient support apparatus 20 associates itself with a particular locator unit 60 that has a non-UWB display device 56 coupled to its video port 144, it is not necessary for the non-UWB display device 56 to be positioned within space volume 152 in order for controller 132 to send data to be displayed on the non-UWB display device 56. Instead, controller 132 is configured to only check whether the associated locator unit 60 is positioned within space volume 152. If it is, controller 132 may send (depending upon the user settings) vital sign data and/or other data to the locator unit 60 for forwarding to the display. If locator unit 60 is not positioned within space volume 152, then controller 132 does not send any data to the locator unit 60 for displaying on non-UWB display device 56, even if the non-UWB display device 56 happens to be positioned within space volume 152.

In addition to allowing a user to customize what data, if any, that is sent to a display device 56, 156, controller 132 is further configured to allow a user to customize what data is sent to different types of display device 56, 156. Thus, controller 132 may be programmed to allow a user to send a first set of data to a non-UWB display device 56 that is coupled to an associated locator unit 60 and a second set of data, different from the first set of data, to a UWB display device 156 that is not coupled to a locator unit 60.

UWB display device 156 may take on a variety of different forms. In some embodiments, display devices 156 may be conventional smart phones, laptop computers, tablet computers, smart TVs and/or smart monitors that include a UWB transceiver built into them, or that have an external tag attached to them with a UWB transceiver and that are able to communicate with the attached UWB tag. It will be appreciated, that in such cases, the conventional smart phone, laptop computer, tablet computer, or other type of UWB display device 156 will include a software app that oversees the UWB communications with patient support apparatus 20 and that controls the display of the data on the display (i.e. screen) of the UWB display device 156. The software app will have one or more security features built into it that only allow authorized users to have this data displayed on the UWB display device 156, thereby preventing unauthorized users from viewing this data on their own smart phone, laptop computer, tablet computer, etc. In some embodiments, controller 132 is configured to execute a security check protocol with the UWB display device 156 to authenticate the UWB display device 156 prior to sending it any data for displaying thereon.

Non-UWB display device 56 (FIG. 5), like UWB display device 156, includes a display controller 162a and a display 164a. Non-UWB display device 56 differs from UWB display device 156 in that it does not include a UWB transceiver 160 built into it, or otherwise coupled to it, such as is found in UWB display devices 156. Because non-UWB display device 56 does not include a UWB transceiver (or multiple UWB transceivers), patient support apparatus 20 is not able to use its UWB transceivers 124 to determine the location of non-UWB display device 56. In contrast, because UWB display devices 156 include one or more UWB transceivers 160, controller 132 of patient support apparatus 20 is able to determine the location of UWB display devices 156 relative to patient support apparatus 20 using ranging, or other position-determining communications, between UWB transceiver(s) 124 and UWB transceiver(s) 160.

In some instances, patient support apparatus 20 is configured to forward data to be displayed to a non-UWB display device 56 by first using UWB transceivers 124 to determine the location of the locator unit 60 to which the non-UWB display device 56 is coupled. If the locator unit 60 is not positioned within the space volume 152, the controller 132 will not send data to be displayed to the locator unit 60 for forwarding to the coupled non-UWB display device 56. If the locator unit 60 is positioned within the space volume, then controller 132 is configured to be able to send data to be displayed on the coupled non-UWB display device 56 by forwarding the data to be displayed to the associated locator unit 60, which then forwards it to non-UWB display device 56. In some embodiments, the locator unit 60 forwards this data to be displayed to the non-UWB display device 56 via video port 144 and video cable 102, such as is shown in FIG. 5. In other embodiments, as will be discussed in more detail below, locator unit 60 may forward the data to be displayed to non-UWB display device 56 using a wireless connection between locator unit 60 and the non-UWB display device 56. The wireless connection may utilize Bluetooth, WiFi, or another wireless technology. In still other embodiments, locator unit 60 may forward the data to be displayed to a non-UWB display device 56 by first forwarding it to the local network 80, such as server 84, which then uses the network to forward it to the non-UWB display device 56. In some embodiments, controller 132 forwards the data to be displayed on non-UWB display device 56 to the associated locator unit 60 by using Bluetooth transceiver 122 to send the data to Bluetooth transceiver 106 of the locator unit 60.

Because non-UWB display device 56 does not include a UWB transceiver, non-UWB display device 56 may be a conventional television, computer monitor, or other conventional device, that is capable of displaying the video signals that are transmitted over video cable 102. In addition, it is not necessary for non-UWB display device 56 to execute any specialized software app that authenticates communication with the coupled locator unit 60 and/or with patient support apparatus 20. Instead, once controller 132 determines that locator unit 60 is within space volume 152 (and, in some cases, determines that locator unit 60 is an authentic locator unit), controller 132 need not perform any additional security protocols regarding non-UWB display device 56. Indeed, controller 132 does not need to be able to perform any communications with non-UWB display device 56 in some embodiments other that forwarding data to be displayed to the associated locator unit 60. In this manner, non-UWB display device 56 need not know anything about locator unit 60 and/or patient support apparatus 20. It merely displays the video signal coming from video cable 102, and therefore does not need a software app that is specialized to patient support apparatus 20 and/or to locator unit 60 and/or to UWB communications.

In some embodiments, patient support apparatus 20 includes a cartridge port 128 and cartridge receiver 126 (FIG. 5). Cartridge port 128 is adapted to physically receive a cartridge 142 via an engineering fit such that cartridge 142 is physically retained on patient support apparatus 20 when cartridge 142 is inserted therein, but that is flexible enough to allow the cartridge 142 to be removed therefrom in response to a user grasping cartridge 142 and pulling on it. In other words, cartridge port 128 is adapted to allow a user to attach cartridge 142 to patient support apparatus 20 and have it remain attached thereto until the user wishes to remove cartridge 142, at which point it is easily removed by the user without using any tools.

Cartridge 142 includes a number of structures that will be described in greater detail below. In general, cartridge 142 is adapted to allow one or more vital sign sensors 140b to be physically plugged into it. That is, as shown in FIG. 5, a vital sign cable 170 may be coupled between vital sign sensor 140b and cartridge 142. When so coupled, the vital sign sensor 140b is adapted to forward its vital sign readings to cartridge 142, which then forwards the readings to cartridge receiver 126. Cartridge receiver 126, in turn, forwards those readings to controller 132 and controller 132 is adapted to display this vital sign data on any one or more of display devices 56 or 156 (or 52) in the manners discussed above. Additionally, or alternatively, controller 132 may forward this data to a patient support apparatus server 84 via network transceiver 96.

By receiving vital sign data through cartridge 142, which is adapted to be physically coupled to patient support apparatus 20, it is not necessary for the location of a vital sign sensor 140b coupled by a cable 170 to cartridge 142 to be determined prior to associating that vital sign sensor 140b with patient support apparatus 20 (or the patient assigned to patient support apparatus 20). Thus, when controller 132 receives vital sign data from cartridge receiver 126, it knows that that vital sign data is from the patient associated with patient support apparatus 20. Controller 132 therefore does not need to use UWB transceivers 124 to determine the location of vital sign sensors 140b in order to associate their data with the patient assigned to patient support apparatus 20. Consequently, vital sign sensors 140b do not need to include a UWB transceiver, whether built into them or integrated into a tag that is attached to the vital sign sensor 140b. Instead, vital sign sensors 140b, as shown in FIG. 5, merely include a vital sign transducer 150 and a controller 148. Controller 148 and transducer 150 may take on any of the same forms, and operate in any of the same manners, described above with respect to controller 148 and transducer 150 of vital sign sensor 140a, which need not be repeated herein.

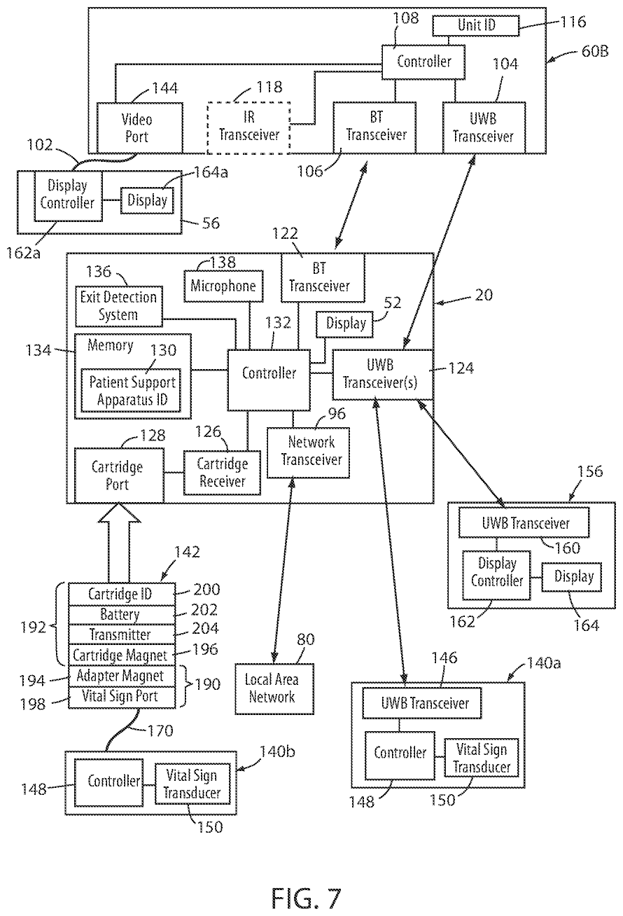

FIGS. 6 and 7 illustrate an unlinked locator unit 60b that functions to provide location information to one or more patient support apparatuses 20 in the same manner as has been described herein (and as is described further below). As was noted previously, unlinked locator units 60b differ from linked locator units 60a in that unlinked locator units 60b are not communicatively coupled to a communications outlet 64. As a result, unlinked locator units 60b can be positioned at any fixed location within the healthcare facility, rather than only at locations that are next to a communications outlet 64. Because such communication outlets 64 are typically only placed along the headwalls within patient rooms, linked locator units 60a are typically only positioned on the headwalls of patient rooms. Unlinked locator units 60b, however, are able to be placed in hallways, in elevators, in maintenance areas, storage areas, parking garages, and/or at any other desired locations where patient support apparatuses 20 are expected to travel and where location information regarding the patient support apparatuses 20 is desired.

FIG. 6 illustrates an unlinked locator unit 60b positioned in a hallway. FIG. 7 illustrates the internal components of locator unit 60b. As can be seen in FIG. 7, locator unit 60b does not include a number of components that may be found in linked locator units 60a, such as configuration circuitry 110, television controller 112, headwall interface 114, and/or IR transceiver 118. Instead, locator unit 60b includes a controller 108, Bluetooth transceiver 106, UWB transceiver 104, a video port 144, and a unit ID 116. These five components work in the same manner as the corresponding components of linked locator units 60a. That is, UWB transceiver 104 is used in conjunction with transceivers 124 to determine the relative position of locator unit 60b with respect to patient support apparatus 20. Controller 108 sends unit ID 116 to patient support apparatus 20, as well as any location and/or angular calculations that it makes in response to the communications between UWB transceiver 104 and UWB transceivers 124. BT transceiver 106 may be used for any high bandwidth communications between locator unit 60b and patient support apparatus 20 that require, or are desirably carried out with, a communication channel having greater bandwidth than the communication channel between UWB transceivers 104 and 124. Such high bandwidth communications may include vital sign data and/or other data that is to be displayed on a non-UWB display device 56 coupled to unlinked locator unit 60b.

Communication between patient support apparatus 20 and an unlinked locator unit 60b takes place in the same manner as was described above with respect to FIGS. 4 and 5. In general, controller 132 of patient support apparatus 20 associates itself with an unlinked locator unit 60b if it is positioned within space volume 152. After associating itself with an unlinked locator unit 60b, it determines (from a signal sent by locator unit 60b) whether a non-UWB display device 56 is coupled to the locator unit 60b and whether it is configured to send any vital sign data, or other data, to be displayed thereon. If it is, controller 132 forwards the data to be displayed on the non-UWB display device 56 to the locator unit 60b which, in turn, forwards it to the display device via its video port 144.

In some embodiments, patient support apparatus 20 may be configured to determine which locator units 60 (linked or unlinked) it is to associate itself with in any of the same manners that the patient support apparatuses and wall units carry out such association in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosure of which is incorporated herein by reference.

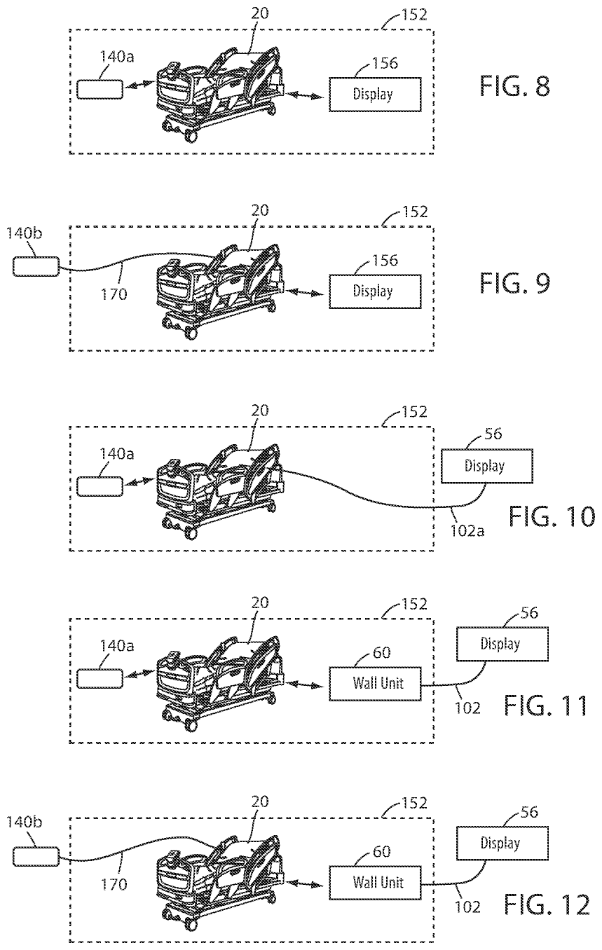

FIGS. 8-16 and 25-26 illustrate a plurality of different operational arrangements for patient support apparatus 20, vital sign sensors 140, display devices 56, 156, and/or locator units 60. That is, each of FIGS. 8-16 and 25-26 illustrate a physical arrangement of one or more of these devices that may be implemented within a healthcare facility at a particular time. Such different arrangement may occur at different times for the various devices, depending upon how the workers at the healthcare facility arrange the devices at any given moment. The manner in which these devices interact with each other in these different operational arrangements will now be discussed in greater detail.

FIG. 8 illustrates a first operational arrangement in which both a vital signs monitor 140a and a UWB display device 156 are positioned inside of the volume of space 152. As was discussed above, patient support apparatus uses its onboard UWB transceivers 124 to determine the location of vital signs monitor 140a and UWB display device 156, checks to see if either or both of them are within space 152, and, in this case, concludes that both of them are within space 152. Because they are both within space 152, controller 132 of patient support apparatus 20 associates these two devices with patient support apparatus 20 (and/or the patient assigned to patient support apparatus 20). As a result, controller 132 is configured to accept vital sign readings from vital sign sensor 140a and to process them in any of the manners discussed above (e.g. display them on UWB display device 156, display them on a display 52 onboard patient support apparatus 20, and/or send them to a server on network 80.

In the situation shown in FIG. 8, controller 132 is configured to disassociate patient support apparatus 20 from vital sign sensor 140a and/or UWB display device 156 if either or both of them move outside of space volume 152. In such instances, if vital sign sensor 140a is moved outside of space 152 but UWB display device 156 remains within space 152, controller 132 no longer associates the vital sign readings from vital sign sensor 140a with a particular patient and stops forwarding them on to other devices, such as UWB display device 156 and/or a server on network 80 (e.g. server 84 and/or an EMR server). To the extent patient support apparatus 20 is forwarding other data to UWB display device 156 for display thereon (e.g. the patient weight from an onboard scale system, the room location of patient support apparatus 20, and/or other information), controller 132 continues to forward that data to UWB display device 156, but ceases to send vital sign information from vital sign sensors 140a to UWB display device 156. If UWB display device 156 is moved outside of space 152 but vital sign sensor 140a remains within space 152, controller 132 stops sending any data to UWB display device 156, but may continue to send data to its onboard display 52 and may also, or alternatively, continue to send vital sign readings from sensor 140a to server 84 and/or an EMR server. If both UWB display device 156 and vital sign sensor 140a move outside of space 152, controller 132 both stops displaying any data on UWB display device 156 and stops forwarding any vital sign data from vital sign sensor 140a anywhere.

Turning to FIG. 9, it illustrates the same general arrangement as FIG. 8 except that the vital sign sensor 140b is coupled to patient support apparatus 20 via a cable, rather than the wireless connection shown in FIG. 8. FIG. 9 therefore illustrates the fact that the location of vital sign sensor 140b relative to space volume 152 is irrelevant because controller 132 automatically associates the readings from vital sign sensor 140b to the patient assigned to patient support apparatus 20 when the vital sign sensor 140b is coupled to the patient support apparatus 20 via cable 170. In other words, when vital sign sensor 140b communicates with patient support apparatus 20 over a cable 170, rather than wirelessly like in FIG. 8, controller 132 uses the physical connection as the trigger for associating the vital sign sensor with patient support apparatus 20, rather than using the vital sign's presence within space volume 152 as the trigger for such association. Thus, in the arrangement shown in FIG. 9, controller 132 is configured to forward vital sign data from vital sign sensor 140b (and in some cases other data) to UWB display device 156 for display thereon, if the user has enabled this type of data display.

FIG. 10 illustrate an operational arrangement wherein a non-UWB display device 56 is coupled directly to patient support apparatus 20 via a video cable 102a and vital sign sensor 140a is positioned inside of space volume 152. Because non-UWB display device 56 is coupled to patient support apparatus 20 via a cable 102a, controller 132 automatically associates non-UWB display device 56 with patient support apparatus 20. Further, because vital sign sensor 140a is positioned inside of space volume 152, controller 132 also automatically associates vital sign sensor 140a with patient support apparatus 20 (using its UWB positioning function). Accordingly, patient support apparatus 20, if configured by the user to do so, sends data from vital sign sensor 140a to non-UWB display device 56 for display thereon (as well as, in some cases, to a server on network 80 and/or a display 52 on patient support apparatus 20).

FIG. 11 illustrates an operational arrangement wherein both a locator unit 60 and a vital sign sensor 140a are positioned inside of space volume 152. In this arrangement, controller 132 is adapted to associate both vital sign sensor 140a and locator unit 60 with patient support apparatus 20. As a result, controller 132 accepts vital sign data from vital sign sensor 140a and may transmit it to locator unit 60 for displaying on non-UWB display device 56, depending upon whether the user has allowed such transmission of vital sign data for display thereon. It should be noted that, as shown in FIG. 11, the position of non-UWB display device 56 relative to space volume 152 is not taken into consideration (indeed it may not be known) by controller 132 when sending data to locator unit 60 for displaying on non-UWB display device 56. Instead, controller 132 only checks to see if locator unit 60 is within space volume 152 and if it is, it sends the vital sign data to locator unit 60 for forwarding to non-UWB display device 56. On the other hand, if non-UWB display device 56 is positioned inside of space volume 152 but locator unit 60 is positioned outside of space volume 152, controller 132 is configured to not send any vital sign data to locator unit 60 for forwarding to non-UWB display device 56.

FIG. 12 illustrates another operational arrangement in which vital sign sensor 140b and non-UWB display device 56 are both positioned outside of space volume 152, and locator unit 60 is positioned inside of space volume 152. In this particular arrangement, because vital sign sensor 140b is coupled to the patient support apparatus 20 by cable 170, controller 132 automatically associates the vital sign sensor 140b with patient support apparatus 20, and therefore accepts these vital sign readings and forwards them to one or more displays and/or to one or more servers on network 80. In addition, because locator unit 60 is positioned inside of space volume 152, controller 132 automatically associates locator unit 60 to patient support apparatus 20. As a result, controller 132 will automatically forward vital sign data to locator unit 60 for display on non-UWB display device 56 if the user has configured patient support apparatus 20 to automatically displaying such vital sign data. As with the arrangement in FIG. 11, the fact that non-UWB display device 56 is positioned outside of space volume 152 in FIG. 12 does not affect the communications between patient support apparatus 20 and locator unit 60.

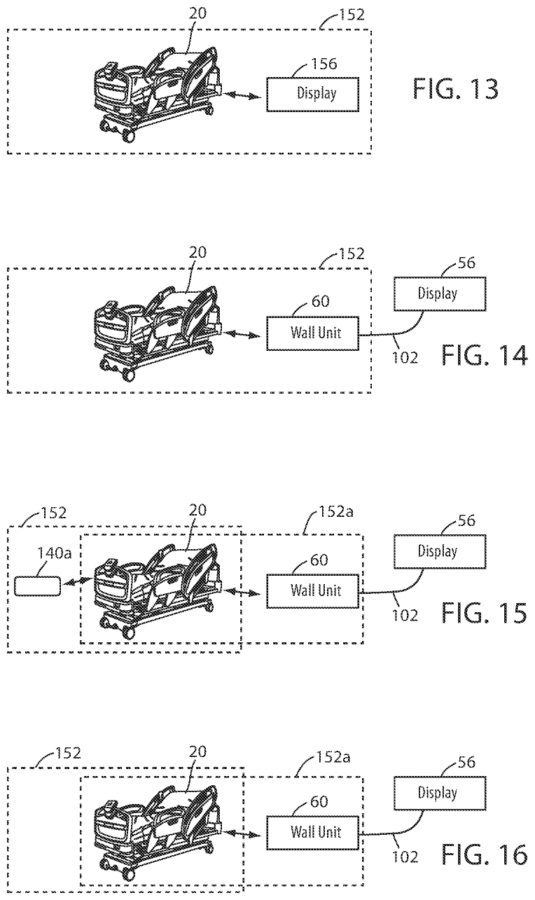

FIG. 13 illustrates an operational arrangement in which no vital sign sensor 140 is present but a UWB display device 156 is positioned inside of space volume 152. In this arrangement, patient support apparatus 20 is configured to be able to automatically forward data for display on UWB display device 156, such as any of the patient support apparatus status data mentioned herein (e.g. patient weight, brake status, height of litter frame 28, status of exit detection system 136, room location, etc.). As with the vital sign data, patient support apparatus 20 is configured to allow a user to change what status data it will automatically share with a UWB display device 156 that enters space volume 152, including not sharing any such data with a UWB display device 156 that is positioned inside of space volume 152.

FIG. 14 illustrates an operational arrangement in which, like FIG. 13, there is no vital sign sensor 140, but there is a locator unit 60 and a non-UWB display device 56 coupled to the locator unit 60. In this arrangement, because locator unit 60 is positioned inside of space volume 152, controller 132 is configured to send patient support apparatus status data, and/or other data, to locator unit 60 for forwarding to non-UWB display device 56. As was noted in several previous arrangements, the forwarding of this data to non-UWB display device 56 is not contingent upon the location of non-UWB display device 56 relative to space volume 152 because non-UWB display device 56 is coupled by cable 102 to locator unit 60, and locator unit 60 is positioned inside of space volume 152 in this arrangement.

As was discussed previously, each space volume 152 is defined with respect to patient support apparatus 20 and therefore moves as patient support apparatus 20 moves. In some embodiments of locator units 60, each locator unit 60 is programmed with the definition of a separate space volume 152a that is fixed with respect to the particular locator unit 60. In such embodiments, controller 132 is configured to use space volume 152 for determining whether a medical device (e.g. a vital sign sensor 140) and/or a UWB display device 156 should be associated with patient support apparatus 20, and to use space volume 152a for determining whether patient support apparatus 20 should be associated with a particular locator unit 60. That is, patient support apparatus 20 is configured to use two different criteria for determining whether to associate a device with patient support apparatus 20—one for locator units 60 and another one for display devices and/or medical devices that are used to treat a patient. In some embodiments, patient support apparatus 20 may utilize additional space volumes 152 beyond these two space volumes. Such additional space volumes 152 may be used for determining whether to associate with other devices. In other words, the particular space volume 152 used by patient support apparatus 20 may vary depending upon the particular device whose position (and association/disassociation with patient support apparatus 20) is being analyzed.

In those embodiments of locator unit 60 that store the definition of a space volume 152a within their onboard memory, the locator unit 60 may be configured to forward this definition to patient support apparatus 20 for controller 132 to use to determine whether patient support apparatus 20 is positioned within the space volume 152a or not. Alternatively, controller 108 of the locator unit 60 may use this definition of the space volume 152a to determine whether patient support apparatus 20 is positioned inside of the space volume 152a or not, and to then forward the result of this determination to patient support apparatus 20. In either scenario, controller 132 is configured to automatically associate itself to the locator unit 60 if it is positioned inside of space volume 152a, or to not associate itself with the locator unit 60 if it is not currently positioned inside of the space volume 152a.

In those embodiments of patient support apparatus 20 that utilize a space volume 152a to determine their association to a locator unit 60, controller 132 is configured to not use space volume 152 for determining this association. That is, controller 132 only uses a single space definition for determining the association, or lack of association, between a patient support apparatus 20 and a locator unit 60. Thus, in such embodiments, controller 132 only uses space volume 152 for determining the association of patient support apparatus 20 with one or more vital sign sensors 140 and/or one or more display devices 156.

FIGS. 15 and 16 illustrate two operational arrangements of a patient support apparatus 20 that uses a space volume 152a for determining its association with a locator unit 60, rather than space volume 152. In FIG. 15, patient support apparatus 20 is positioned inside of a space volume 152a that is defined in a fixed manner with respect to locator unit 60. As a result, controller 132 of patient support apparatus 20 automatically associates that locator unit 60 with patient support apparatus 20. A vital sign sensor 140a is also shown in FIG. 15 and positioned inside of space volume 152, but not inside of space volume 152a. Because it is positioned inside of space volume 152, controller 132 automatically associates it with patient support apparatus 20. Space volume 152a is not used by controller 132 to determine the association, or lack of association, between patient support apparatus 20 and vital sign sensor 140a. Similarly, space volume 152 is not use by controller 132 to determine the association, or lack of association, between patient support apparatus 20 and locator unit 60. In the arrangement shown in FIG. 15, controller 132 is configured to be able to automatically forward vital sign readings from vital sign sensor 140a to UWB display device 156 (via locator unit 60).

FIG. 16 illustrates another operational arrangement of patient support apparatus 20, locator unit 60, and non-UWB display device 56, wherein a space volume 152a is defined in a fixed relationship with locator unit 60 (and space volume 152 is defined in fixed relationship to patient support apparatus 20). In this particular arrangement, patient support apparatus 20 is positioned inside of space volume 152a and controller 132 therefore automatically associates patient support apparatus 20 with locator unit 60. No vital signs sensors 140, or other medical devices, are positioned inside of space volume 152, so controller 132 does not associate any vital sign sensors, or other medical devices, with patient support apparatus 20. Accordingly, patient support apparatus 20 does not have any vital sign data, or data from other types of medical devices, to forward to locator unit 60 for display on non-UWB display device 56. However, as was noted previously, patient support apparatus 20 may be configured by a user to display status data regarding patient support apparatus 20 on non-UWB display device 56, and/or other data. If a user has so configured patient support apparatus 20, controller 132 may forward, for example, its room location, its brake status, its exit detection system status, etc. to locator unit 60 for display on non-UWB display device 56. As was mentioned in several of the previously discussed arrangements, the fact that non-UWB display device 56 is positioned outside of space volume 152a (and/or 152) in the FIG. 16 arrangement does not prevent controller 132 from forwarding data to locator unit 60 for display on non-UWB display device 56 because patient support apparatus 20 is positioned inside of space volume 152a, and it is the association between patient support apparatus 20 and locator unit 60 that determines whether data for display is forwarded to locator unit 60, not an association between patient support apparatus 20 and non-UWB display device 56.

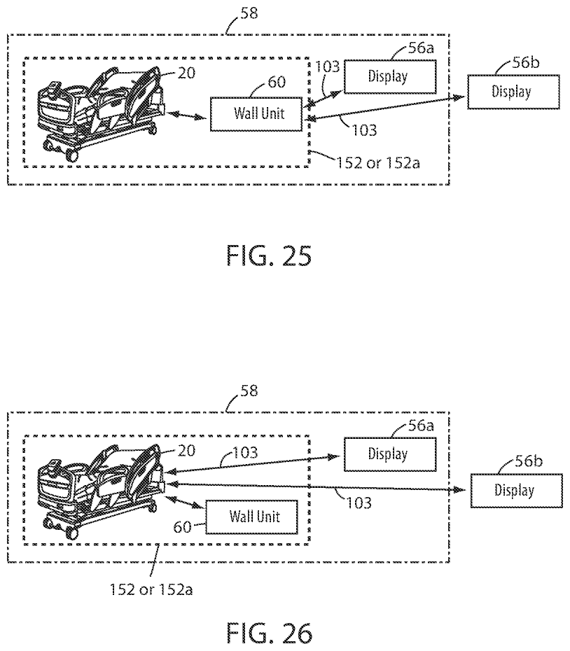

FIG. 25 illustrates another operational arrangement of patient support apparatus 20, locator unit 60, and two non-UWB display devices 56. A first one of the non-UWB display devices 56a is positioned inside the same room 58 as patient support apparatus 20 while a second one of the non-UWB display devices 56b is positioned outside of the room 58, such as, but not limited to, in the hallway outside of the room 58. In the operational arrangement of FIG. 25, patient support apparatus 20 is positioned inside of space volume 152 (or 152a) and controller 132 therefore automatically associates patient support apparatus 20 with locator unit 60. Non-UWB display devices 56a and 56b are communicatively coupled to locator unit 60 by wireless links 103, rather than by video cable 102. Locator unit 60 is configured in this embodiment to forward the data to be displayed that it receives from patient support apparatus 20 to either or both of non-UWB display devices 56a, 56b via the wireless links 103. As with the other operational arrangements discussed herein, the forwarding of data to be displayed on non-UWB display devices 56a and/or 56b may commence automatically upon patient support apparatus 20 moving within space volume 152 (or 152a) and associating with locator unit 60 (which informs patient support apparatus 20 of the existence of the non-UWB display devices 56a and 56b to which it is communicatively coupled). Patient support apparatus 20 sends the data to be displayed to locator unit 60 which then forwards it to non-UWB display devices 56a and/or 56b.

The transmission of the data to be displayed from patient support apparatus 20 to locator unit 60 may utilize Bluetooth communications between Bluetooth transceivers 122 and 106, or it may utilize UWB communications between UWB transceiver(s) 124 and UWB transceiver 104, or it may utilize another wireless communication technology (e.g. WiFi). The forwarding of the data to be displayed from locator unit 60 to non-UWB display devices 56a and/or 56b may utilize Bluetooth communications between Bluetooth transceiver 106 and a Bluetooth transceiver incorporated into non-UWB display devices 56a and 56b, or it may utilize WiFi communications between locator unit 60 and non-UWB display devices 56a and 56b, or it may utilize other wireless communications.

In some embodiments, controller 108 of locator unit 60 is informed of the addresses of non-UWB display devices 56a and/or 56b during the initial installation of locator unit 60 in the healthcare facility (or at a time subsequent thereto). In such embodiments, the addresses of non-UWB display devices 56a and 56b are saved in a memory onboard locator unit 60. The saving of the addresses of the specific display devices 56 that are associated with a specific locator unit 60 is particularly useful in situations where the display devices 56 are mounted at fixed locations within the healthcare facility and are always to be associated with a particular locator unit 60. In other embodiments, the address of the specific non-UWB display devices 56a and/or 56b associated with a particular locator unit 60 are stored in patient support apparatus server 84, and these addresses are communicated by WiFi to locator unit 60 (in which case it is modified to include a WiFi transceiver), or they are communicated to patient support apparatus 20 by network transceiver 96 and patient support apparatus 20 then forwards them to locator unit 60 via Bluetooth transceiver 122 and/or UWB transceiver(s) 124. Still other means or methods of informing locator unit 60 of the addresses of non-UWB display devices 56a and/or 56b may be used.

In some embodiments, the addresses are IP addresses and/or include additional information (e.g. subnet masks, SSID, password, etc.). In other embodiments, the address may include other address information, such as a Media Access Control (MAC) address, a Bluetooth address, etc. Still other type of information may be included that enables the locator unit 60 to address the data to be displayed to the intended non-UWB display devices 56a and/or 56b. It will be understood that the term “address” may refer to any identifier of a recipient display device 56 that allows a sender (e.g. locator unit 60) to send information to that particular display device 56.

FIG. 26 illustrates another operational arrangement of patient support apparatus 20, locator unit 60, and two non-UWB display devices 56. FIG. 26 is similar to FIG. 25 in that it includes a first non-UWB display device 56a positioned inside the same room 58 as patient support apparatus 20 and a second non-UWB display device 56b positioned outside of the room 58. The operational arrangement of FIG. 26 differs from that of FIG. 25 in that, instead of sending data to be displayed from patient support apparatus 20 to locator unit 60, which then forwards it to display devices 56, as in FIG. 25, the operational arrangement of FIG. 26 has the patient support apparatus 20 sending the data to be displayed directly to non-UWB display devices 56a and 56b, thereby bypassing locator unit 60. In other words, in the operational arrangement of FIG. 26, locator unit 60 does not operate as an intermediate recipient of the data to be displayed on non-UWB display devices 56a and 56b. Instead, non-UWB display devices 56a and 56b receive the data for display directly from patient support apparatus 20 (or, in some embodiments, from a connection to network 80).

Patient support apparatus 20 may be informed of the addresses associated with non-UWB display devices 56a and/or 56b in any of the same manners discussed above with respect to FIG. 25 and locator unit 60. That is, controller 132 of patient support apparatus 20 may be informed of the addresses of the specific non-UWB display devices 56a and/or 56b associated with a specific locator unit 60 when patient support apparatus 20 is initially installed within a healthcare facility (or at a time subsequent thereto). In such embodiments, the addresses of non-UWB display devices 56a and 56b are saved in a memory 134. In other embodiments, the addresses associated with non-UWB display devices 56a and 56b are stored in a memory onboard locator unit 60 and locator unit 60 automatically forwards these addresses to patient support apparatus 20 after patient support apparatus 20 and locator unit 60 are associated with each other. In other embodiments, the addresses of the specific non-UWB display devices 56a and/or 56b associated with a particular locator unit 60 are stored in patient support apparatus server 84, and these addresses are communicated by WiFi to patient support apparatus 20 via network transceiver 96. In such embodiments, patient support apparatus 20 may send a query to patient support apparatus server 84 that includes the unique ID 116 of the locator unit 60 with which it is currently associated, and patient support apparatus server 84 may thereafter respond with the address(es) of the specific non-UWB display devices 56a, b that are associated with the locator unit 60 having that particular ID 116.

Although FIGS. 13-14, 16, and 25-26 do not depict a vital sign sensor 140, it will be understood that one or more vital sign sensors 140, and/or one or more other devices may be included in these operational arrangements, and such vital sign sensors 140, or other devices, may have their relative position to patient support apparatus 20 automatically determined by patient support apparatus 20. If the vital sign sensor 140, or other device(s), are positioned inside of space volume 152, data from one or more of these devices may be automatically communicated to patient support apparatus 20 for forwarding to a non-UWB display device 56 for display thereon. Alternatively, or additionally, data from patient support apparatus 20 itself may be forwarded to one or more of these non-UWB display devices 56.

It will also be understood that, although FIGS. 8-16 depict only a single display device 56, and that although FIGS. 25 and 26 depict two non-UWB display devices 56a and 56b, the number of display devices 56 in any of these figures can be changed from what is shown therein. Thus, for example, either of the operational arrangements of FIGS. 25 and 26 may include only a single non-UWB display device 56, or they may include three or more non-UWB display devices 56. Similarly, any of the operational arrangements of FIGS. 8-16 may include more than a single non-UWB display device 56.

It will also be understood that because a room 58 is not shown in FIGS. 8-16, the location of the components shown therein relative to a room 58 can vary. That is, for example, in FIG. 10, display 56 can be located inside of a room 58 in which the patient support apparatus 20 is located, or it may be located outside of a room 58 in which the patient support apparatus 20 is located. Similarly, because no room is shown in FIG. 10, patient support apparatus 20 may itself be positioned outside of a room 58 (e.g. such as in a hallway). Further, it will also be understood that, although FIGS. 25 and 26 depict patient support apparatus 20 within a room 58, it is not necessary that patient support apparatus 20 be positioned within a room 58 to implement the operational arrangements shown therein. Thus, for example, in the arrangement shown in FIG. 25, locator unit 60 could be an unlinked locator unit 60b, patient support apparatus 20 may be positioned in a hallway (or elsewhere outside of a room 58), and non-UWB display devices 56a and/or 56b can be positioned at any locations that are within communication range of the locator unit 60 shown therein.

Still further, it will be understood that, although non-UWB display devices 56 and UWB-display devices 156 have been described herein as primarily serving to display data from patient support apparatus 20 and/or any devices associated with patient support apparatus 20, display devices 56 and/or 156 may be configured to receive data from a user and forward that data to patient support apparatus 20 and/or to a device associated with patient support apparatus 20. Thus, display devices 56 and/or 156 may be used to communicate with and/or to control various aspects of patient support apparatuses 20 and/or their associated devices. In some such embodiments, display devices 56 and/or 156 may include a touchscreen that is used by caregivers to send data and/or commands to the associated patient support apparatus 20 (and/or its associated devices). Display devices 56 and/or 156 may therefore act as remote controls for their associated patient support apparatus 20 and/or the device(s) associated with that particular patient support apparatus 20.

In some situations, one or more display devices 56 and/or 156 may be positioned outside of individual rooms 58 within a healthcare facility, such as near the doorway to such rooms, or elsewhere. When so positioned, a caregiver would be able to not only see the status of the patient support apparatuses 20 (and/or associated devices) within the rooms without having to go inside of the rooms 58, but would also be able to control the patient support apparatuses 20 (and/or associated devices) without having to go inside of the rooms 58. Additional displays 56 and/or 156 may also be positioned within the rooms 58, and/or in other locations, making the status of the patient support apparatuses 20 more visible to caregivers.

Although FIGS. 8-16 and 25-26 depict a variety of different arrangements in which patient support apparatus 20 is adapted to operate, it should be understood that these are merely a representative sampling of the types of arrangements in which patient support apparatus 20 is configured to operate. Patient support apparatus 20 is adapted to operate in arrangements besides those explicitly shown in FIGS. 8-16 and 25-26. For example, patient support apparatus 20 may be operated with more than the single vital sign sensor 140 shown in FIGS. 8-12. That is, multiple vital sign sensors 140 may be in use on a particular patient and controller 132 of patient support apparatus 20 is adapted to determine the locations of those multiple vital sign sensors relative to space volume 152, and, if they are positioned inside of space volume 152, to automatically associate them with patient support apparatus 20 and make their data available for display and/or forward their data to computer network 80. As another example, multiple display devices 56 and/or 156 may be utilized with patient support apparatus 20 and the same, or different, data may be automatically displayed by controller 132 on these displays.

As yet another example, although FIGS. 8-12 and 15 depict only a vital sign sensor 140 associated with patient support apparatus 20, it will be understood that patient support apparatus 20 is adapted to automatically associate itself with a variety of other types of medical devices besides vital sign sensors whenever those other medical devices are positioned inside of space volume 152. Indeed, in some embodiments, controller 132 is adapted to automatically associate itself with any one or more of the devices disclosed in commonly assigned U.S. patent application 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosure of which is incorporated herein by reference. Patient support apparatus 20 is configurable by a user to also, or alternatively, automatically display any of the data from these other types of devices on any of the display devices 56 and/or 156 disclosed herein when these devices are associated with patient support apparatus 20 and one or more display devices 56 and/or 156 (or a connected locator unit 60) are also associated with patient support apparatus 20. The devices with which patient support apparatus 20 may associate itself using UWB transceiver(s) 124 include, but are not limited to, any one or more of the following: one or more exercise devices, heel care boots, IV stands and/or poles, infusion pumps, DVT pumps, overhead booms used in operating rooms and/or other healthcare facility room, ventilators, patient sensors (e.g. saturated oxygen (SpO₂) sensors, EKG sensors, vital sign sensor, etc.), patient positioning devices (e.g. wedges, turning devices, pumps), ambient sensors (e.g. air temperature, air flow, light, humidity, pressure, altitude, sound/noise), mattresses 42, incontinence pads or one or more sensors adapted to detect patient incontinence, Holter devices adapted to monitor and record a patient's heart signals, patient ID tags or bracelets adapted to be worn by patients, caregiver tags or ID bracelets adapted to be worn by caregivers, pieces of furniture, and/or other types of devices), and/or other devices.

It should also be noted that the display of vital sign data, or other device data, on one or more of display devices 56 and/or 156 is carried out by patient support apparatus 20 automatically. That is, for example, when a user has configured patient support apparatus 20 to display a patient's heart rate on a display device 56 and/or 156, it automatically forwards the heart rate data to the display device 56, 156 after the corresponding association has been made (e.g. patient support apparatus 20 has been associated with a UWB display device 156 or a locator unit 60 to which a non-UWB display device 56 is coupled). Consequently, if a patient with a heart rate monitor is wheeled into a bay area of a patient room that includes a fixed non-UWB display device 56 coupled to a locator unit 60, controller 132 is configured to automatically start displaying the patient's heart rate information on that non-UWB display device 56 as soon as it completes the association process with the corresponding locator unit 60. In this manner, the caregiver sees the patient's heart rate displayed on the non-UWB display device 56 within seconds after moving the patient support apparatus 20 into the bay area, and the caregiver doesn't need to connect any cables, press any buttons, or take any other actions, in order for the heart rate data to be displayed on non-UWB display device 56. Similarly, when the patient support apparatus 20 moves out of the bay area, the display of the patient's data on the non-UWB display device 56 automatically terminates.

As another example of the automatic display of medical device data (e.g. vital sign data) on a display, if a caregiver approaches a patient support apparatus 20 while carrying a portable electronic device—such as a smart phone or tablet computer that includes a display, a UWB transceiver, and the appropriate software app—controller 132 is adapted to automatically start forwarding the patient's heart rate data to the portable electronic device for display on its screen. In this manner, a caregiver that brings his or her portable electronic device within space volume 152 can have that patient's heart rate data (and/or other data) automatically displayed on his/her device. When the caregiver moves his or her portable electronic device outside of the space volume 152, the display of that patient's data automatically ceases because controller 132 automatically disassociates patient support apparatus 20 from the portable electronic device and stops sending it data to be displayed. If the caregiver then moves to another patient's patient support apparatus 20 and steps inside the space volume 152 of that patient support apparatus, he or she will automatically see the data from the medical devices associated with that patient displayed on his or her portable electronic device.

The automatic display of data on one or more display devices 56, 156 is able to be carried out by controller 132 because it repetitively determines the relative location of patient support apparatus 20 to vital signs sensors 140 (and/or other devices), to locator units 60, and to UWB display devices 156. In addition, when it detects that one or more of these devices are within communication range, it automatically determines whether they are positioned within space volume 152 (or, in some cases, space volume 152a or another space volume) and, if they are, it automatically associates them to patient support apparatus 20. Once this association is made, the process of forwarding information for display on display device 56 and/or 156 is carried out automatically. It will be understood that the frequency at which controller 132 repetitively determines the relative location of vital sign sensors 140 (or other devices), locator units 60, and/or UWB display devices 156 may vary in different embodiments. In some embodiments, this occurs multiple times a second. In other embodiments, this occurs less frequently than once a second. In still other embodiments, the frequency at which controller 132 determines the relative position of a device to patient support apparatus 20 may vary according to the device, the current location of patient support apparatus 20, the movement state of patient support apparatus 20 (i.e. whether it is currently moving or stationary), the brake state of patient support apparatus 20 (i.e. whether the brake is on or off), and/or according to one or more other factors.

It should also be understood that FIGS. 8-16 and 25-26 do not illustrate different embodiments of patient support apparatuses 20, vital sign sensors 140, locator units 60, and/or display devices 56, 156. Instead, these figures illustrate the same embodiments, but with different physical arrangements and communication paths for data that is to be displayed on one or more display devices 56, 156. However, it will also be understood that any of the embodiments of patient support apparatuses 20, locator units 60, display device 56, 156, and/or vital sign sensors 140 may be used with any of the operational arrangements shown in FIGS. 8-16.

In some embodiments, one or more of the non-UWB display devices 56 may be adapted to dock and undock with a UWB-equipped docking station. In such embodiments, the caregiver can grab and remove the display device from the docking station when he or she wishes to use it (e.g. such as when he/she walks into a patient's room 58), and then replace the display device 56 on the docking station when he or she is done using it. In such embodiments, the docking station may include a UWB transceiver that patient support apparatus 20 associates and disassociates itself with depending upon their relative position to each other. In other embodiments, display devices 56 do not include docking stations and separate removable units, but instead comprise a single integrated structure.

In those embodiments of patient support apparatus 20 where controller 132 is configured to associate itself with locator units 60 based on space volume 152a, rather than space volume 152, locator units 60 may be configured to send the dimensions of space volume 152a to the patient support apparatus 20 for controller 132 to use when determining whether to associate with that locator unit 60 or not. Alternatively, or additionally, patient support apparatus 20 may store in its memory 134 the dimensions of space volume 152a for each locator unit 60. As yet another alternative, controller 132 may not need to know the dimensions of space volume 152a at all, but instead may await a message sent by locator unit 60 to patient support apparatus 20 indicating whether the patient support apparatus 20 should be associated with the locator unit 60 based on locator unit 60's own determination of whether the patient support apparatus 20 is currently positioned within the space volume 152a or not.

It will also be understood that in any of the embodiments of patient support apparatus 20 discussed herein, the size, shape, location, and/or other aspects of space volumes 152 and/or 152a may vary based on one or more factors. For example, in some embodiments, the space volume 152a associated with linked locator units 60a may be different from the space volume 152a associated with unlinked locator units 60b. Additionally, or alternatively, individual ones of linked locator units 60a may have space volumes 152a of different sizes and/or shapes from other individual linked locator units 60a, and/or individual ones of unlinked locator units 60b may have space volumes 152a of different sizes and/or shapes from other individual unlinked locator units 60b.

In some embodiments, patient support apparatus 20 is configured to consult memory 134 to determine the size, shape, and/or other information about a particular space volume 152a. In such embodiments, controller 132 uses the unique ID 116 it receives from the locator unit 60 to determine the size, shape, and/or other information about the space volume 152a. That is, memory 134 contains data correlating particular space volumes 152a to particular locator unit IDs. In this manner, each locator unit 60 can potentially have different space volumes 152a associated with it, and these definitions of these space volumes 152a need not be transmitted from the locator unit 60 to patient support apparatus 20. Instead, controller 132 consults its onboard memory 134 to determine the boundaries of the space volume 152a and decide whether patient support apparatus 20 is currently positioned inside or outside of it.

It will also be understood that in any of the embodiments of patient support apparatus 20 discussed herein, the size, shape, location, and/or other aspects of space volumes 152 and/or 152a may be changed by controller 132 when determining whether to disassociate a device, rather than to associate a device. In other words, once a device has been determined to be positioned inside of a space volume 152, 152a, controller 132 may increase the size of—and/or otherwise change one or more dimensions of—the space volume 152, 152a when determining whether to disassociate the device. In this manner, space volumes 152 and/or 152a may have a sort of hysteresis aspect wherein a device has to be positioned inside of a smaller space volume 152, 152a in order to be associated with another device, but thereafter can only be disassociated if it moves outside of a larger sized space volume 152, 152a. In still other embodiments, the dimensions of space volumes 152, 152a are the same for both association and disassociation purposes.

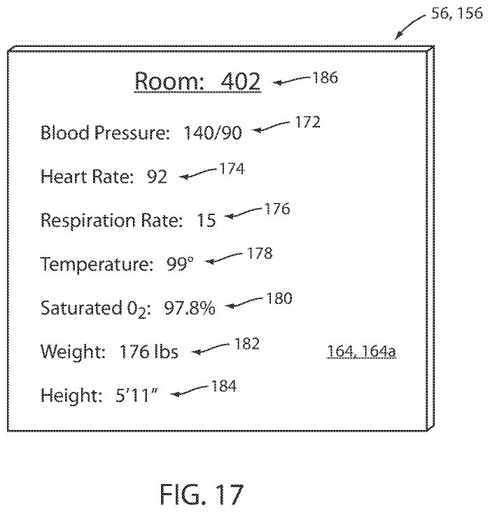

FIG. 17 illustrates one example of the type of data that may be displayed on a display device 56, 156. As shown therein, the display device 56, 156 is displaying a patient's blood pressure 172, the patient's heart rate 174, the patient's respiration rate 176, the patient's temperature 178, the patient's saturated oxygen level 180, the patient's weight 182, and the patient's height 184. UWB display device 156 is also showing the room number 186 in which patient support apparatus 20 is currently located. As was discussed previously, in some embodiments, controller 132 is configured to determine the current location of patient support apparatus 20 after it receives the unique ID 116 from a locator unit 60, forwards that unique ID 116 along with its own unique ID 130 (FIGS. 5 & 7) to patient support apparatus server 84, and server 84 sends a return message back to the patient support apparatus 20 (via access points 82 and network transceiver 96) that informs the patient support apparatus 20 of the location within the healthcare facility corresponding to that particular patient support apparatus 20.

As was also previously mentioned, patient support apparatus 20 includes one or more screens that are displayable on display 52 that allow a user to customize the data that is shown on display device 56 and/or 156. In other words, the user can configure patient support apparatus 20 to change the example of displayed data shown in FIG. 17. In addition, patient support apparatus 20 is configured to allow the user to select what data, if any, from the associated vital sign sensors 140 or other medical devices, is displayed on onboard display 52. Patient support apparatus 20 may also allow the user to change the format and/or layout in which the selected data is to be displayed.

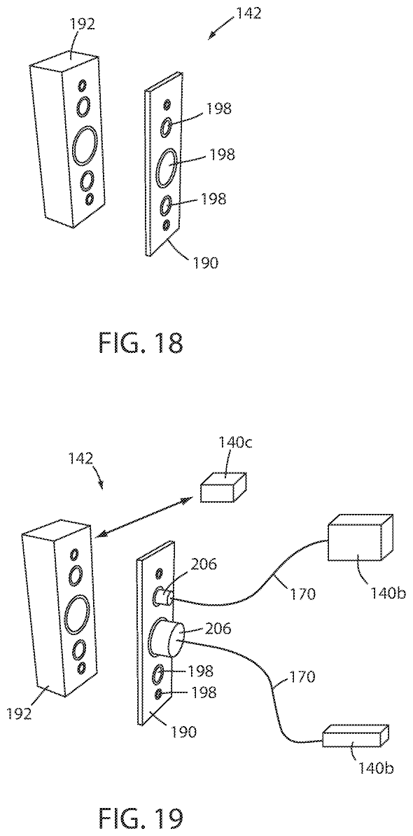

FIGS. 18 & 19 illustrate in greater detail one example of a cartridge 142 that is utilized with some embodiments of patient support apparatus 20. As shown therein, cartridge 142 includes an adapter 190 and a main body 192. Main body 192 and adapter 190 are adapted to be releasably secured together via one or more magnets. That is, as shown in FIGS. 5 & 7, adapter 190 includes one or more adapter magnets 194 that are adapted to magnetically couple to one or more cartridge magnets 196 that are integrated into main body 192 of cartridge 142. The magnets 194 and 196 magnetically secure together main body 192 and adapter 190 when they are positioned in close proximity to each other, but also allow a user to physically separate adapter 190 from main body 192 by pulling them apart.

As is shown in FIGS. 5 and 7, adapter 190 also includes one or more vital sign ports 198, and main body 192 also includes a cartridge ID 200, a battery 202, and a transmitter 204. All or a portion of main body 192 is physically constructed such that it can be selectively retained within cartridge port of 128. In some embodiments, main body 192—or a portion thereof—is selectively maintained in cartridge port 128 of patient support apparatus 20 by way of an engineering fit that is designed to keep cartridge 142 attached to port 128 when no user is pulling on cartridge 142, but to allow cartridge 142 to be removed from cartridge port 128 when a user applies a pulling force to cartridge 142. The pulling force necessary to remove cartridge 142 from port 128 can vary, but may generally be on the order of a few pounds of force, and should be selected such that ordinary caregivers are able to apply the selected force to cartridge 142 without a burden.

As can be seen more clearly in FIGS. 18-19, adapter 190 includes a plurality of vital sign ports 198. Each vital sign port 198 is adapted to physically connect to a cord 170 that, at its other end, is coupled to a vital sign sensor 140. That is, as shown in FIG. 19, a plurality of vital sign sensors 140b are shown coupled to adapter 190 by way of cords 170. Each cord 170 includes a connector 206 at its end that is adapted to mate with one or the vital sign ports 198 on adapter 190. When adapter 190 is physically coupled to main body 192 via magnets 194 and 196, the electrical signals received at vital sign ports 198 of adapter 190 are communicated to main body 192, which then forwards them to cartridge receiver 126 of patient support apparatus 20 via transmitter 204. Cartridge receiver 126, in turn, forwards them to controller 132 which, as explained above, may forward them to one or more displays and/or to network 80.

Adapter 190 (FIGS. 18-19) may be configured to accept different types of connectors 206 from different types of vital sign sensors 140b. However, adapter 190 is configured to physically connect to the same main body 192 of cartridge 142. In this manner, differently styled adapters 190 can be used with the same main body, thereby allowing different types of vital sign sensors to be used with cartridge 142 without having to change main body 192 of cartridge 142. Instead, a user can select whichever adapter 190 is compatible with a particular vital sign sensor 140, or plurality of vital sign sensors 140, plug their cable(s) into the compatible adapter 190, and then couple adapter 190 to main body 192 of cartridge 142.

Cartridge 142 is adapted to allow a patient to easily exit patient support apparatus 20 without requiring the patient, or his or her caregiver, to unplug any cables before the patient is allowed to exit. Instead, the cartridge 142 can simply be pulled out of cartridge port 128 and carried with the patient as he or she exits from patient support apparatus 20. As will be discussed in greater detail below, cartridge 142 is configured, in at least some embodiments, to wirelessly transmit the patient's vital sign readings to patient support apparatus 20 while cartridge 142 is physically unplugged from patient support apparatus 20. In this manner, the patient's vital signs can still be displayed and/or sent to an EMR server on network 80 (or elsewhere), even when the patient has exited patient support apparatus 20.

Cartridge transmitter 204 of cartridge 142 transmits the patient's vital signs to cartridge receiver 126 when cartridge 142 is physically removed from cartridge port 128. In some embodiments, cartridge transmitter 204 includes a Bluetooth transmitter, although it may include a different type of wireless communication transmitter. When cartridge transmitter 204 is implemented as a Bluetooth transmitter, it may communicate with BT transceiver 122 onboard patient support apparatus 20 instead of cartridge receiver 126. In such embodiments, cartridge receiver 126 may be configured to only receive wired communications from cartridge 142, rather than both wired and wireless communications. Cartridge transmitter 204, in addition to wirelessly communicating with patient support apparatus 20, is also adapted to communicate with patient support apparatus 20 in a wired manner when it is physically coupled to patient support apparatus 20 (i.e. when it is inserted into cartridge port 128).

When cartridge 142 is physically connected to patient support apparatus 20, cartridge transmitter 204 communicates its unique cartridge ID to cartridge receiver 126. Controller 132 uses this cartridge ID 200 to associate that particular cartridge 142 with the patient assigned to patient support apparatus 20. In this manner, if cartridge 142 is subsequently removed from cartridge port 128 and begins wirelessly transmitting vital sign data to patient support apparatus 20, controller 132 will be able to assign those wireless readings to the patient assigned to patient support apparatus 20. This is helpful in situations where another cartridge 142 from a second patient support apparatus 20 may be positioned within communication range of the first patient support apparatus 20. In this situation, the first patient support apparatus 20 may receive vital sign signals from two different cartridges 142. Because each cartridge 142 is configured to transmit its unique ID 200 with each wireless message, controller 132 of the first patient support apparatus 20 ignores the wireless messages from the second cartridge 142 because they do not contain the unique ID 200 that it has associated with first patient support apparatus 20. The unique ID 200 is therefore used by controller 132 to distinguish between multiple cartridges 142 and to only process the signals that is receives from the specific cartridge 142 that it has associated with the patient support apparatus 20 it is part of.

In some embodiments, cartridges 142 are adapted to communicate wirelessly with one or more vital sign sensors 140c (FIG. 19). In these embodiments, vital sign sensors 140c include a wireless transceiver that may be a UWB transceiver, or that may be another type of wireless transceiver. In such embodiments, the wireless vital sign sensors 140c may be associated to a particular cartridge 142 in a plurality of different ways. These manners include, but are not limited to, a manual process, a UWB process, and/or a patient support apparatus process. Each of these are explained in greater detail below.

Wireless vital sign sensor 140c (FIG. 19) may be associated with a particular cartridge 142, in some embodiments, through a manual process undertaken by a caregiver that informs cartridge 142 that a particular vital sign sensor 140c is to be associated with it. This manual process can take on a variety of different forms, such as, for example, a pairing process that involves the pressing of one or more buttons on vital sign sensor 140c and/or cartridge 142. It may also, or alternatively, involve a bar code scanner and/or a near field identification reader built into cartridge 142 that reads a unique bar code, or a near field unique identification, from the vital sign sensor 140c when the vital sign sensor 140c is physically positioned next to the scanner or reader. As yet another alternative, patient support apparatus 20 may include a bar code scanner or near field reader built into it, and the user can associate a particular cartridge 142 with a particular vital sign sensor 140c by scanning a bar code, or near field identifier, from both the particular cartridge 142 and the vital sign sensor 140c. In such embodiments, cartridge receiver 126 may be configured to include the ability to transmit data to cartridge 142 (as well as receive it), and cartridge receiver 126 may then transmit to it the unique ID of the vital sign sensor 140c that patient support apparatus 20 scanned. Vital sign sensor 140c includes that unique ID in its wireless messages, and cartridge 142 is therefore able to use that unique ID to differentiate messages from that particular vital sign sensor 140c from other vital sign sensors 140c that may be positioned within communication range. Still other manual association processes are possible.

Wireless vital sign sensor 140c (FIG. 19) may also, or alternatively, be associated with a particular cartridge 142, in some embodiments, by utilizing UWB transceivers that are built into each device. That is, in some embodiments of cartridge 142 and vital sign sensors 140c, each device includes a UWB transceiver built therein (or incorporated into a tag attached to the device). The UWB transceivers are adapted to determine their distance from each other. In such embodiments, cartridge 142 is adapted to automatically associate itself with a particular vital sign sensor 140c if the vital sign sensor 140c is positioned within a threshold distance of it. The threshold distance may vary depending upon the particular location of cartridge 142 when it is attached to patient support apparatus 20, but generally should be selected so as to not encompass vital sign sensors 140c that are positioned on another patient support apparatus 20. The threshold distance, therefore, may be on the order of one or two meters, although other distances may, of course, be utilized.

Wireless vital sign sensor 140c (FIG. 19) may also, or alternatively, be associated with a particular cartridge 142, in some embodiments, by associating the vital sign sensor 140c with the patient support apparatus 20 to which cartridge 142 is also associated. In such embodiments, vital sign sensor 140c may be manually paired with patient support apparatus 20 in any of the manual methods discussed above, after which patient support apparatus 20 informs cartridge 142 of the unique ID of the vital sign sensor 140c that it has been paired with. In other embodiments, vital sign sensor 140c may include a UWB transceiver built into it, or incorporated into an attached tag, and controller 132 may automatically associate the vital sign sensor 140c to patient support apparatus 20 if it is positioned inside space volume 152. Still other methods are possible for associating the vital sign sensor 140c to patient support apparatus 20.

Vital sign sensors 140a, 140b, and 140c may differ from each other with respect to their components. In some embodiments, vital sign sensors 140a may include a UWB transceiver 146 while vital sign sensors 140b and/or 140c do not. Vital signal sensors 140b are adapted to couple to cartridge 142 by way of a cable 170, while vital sign sensors 140a and/or 140c are not so adapted. Further, vital sign sensors 140c may include a non-UWB wireless transceiver in them for communicating wirelessly with cartridge 142, while vital sign sensors 140a and/o 140b may not include such a non-UWB wireless transceiver. Still other variations between the vital sign sensors 140a-c may also be possible.

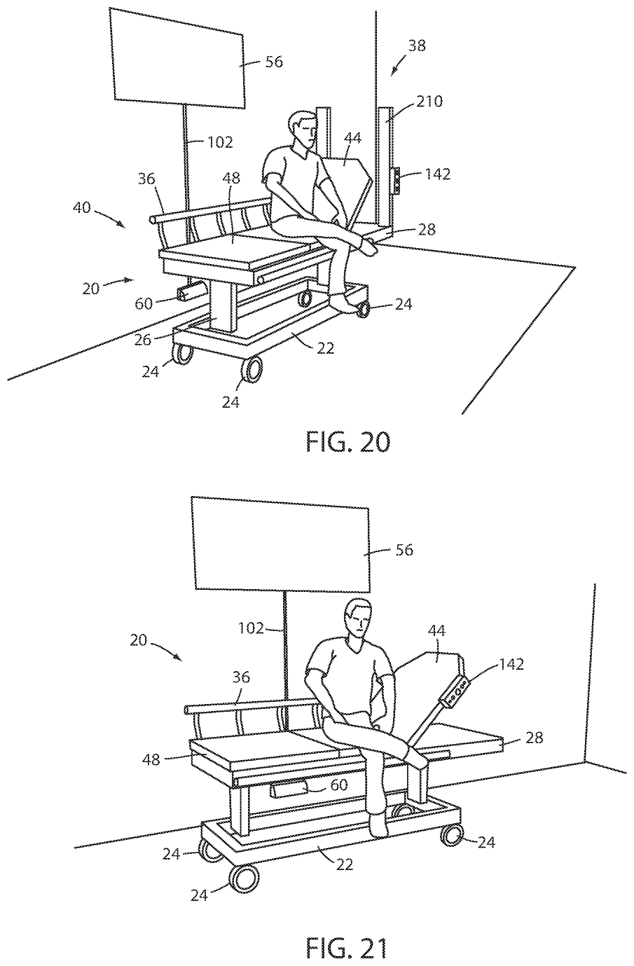

The location at which cartridge 142 may be coupled to patient support apparatus 20, and therefore the location of cartridge port 128, may vary in different embodiments of patient support apparatus 20. FIGS. 20-23 illustrate different locations at which cartridge 142 may be coupled to cartridge port 128 on different embodiments of patient support apparatus 20. FIG. 20 illustrates cartridge 142 coupled to patient support apparatus 20 along a vertical support 210 positioned adjacent head end 38. In this embodiment of patient support apparatus 20, cartridge port 128 is integrated into the vertical support 210. When cartridge 142 is inserted into port 128, cartridge receiver 126 (which may also be positioned inside of vertical support 210 or elsewhere on patient support apparatus 20) is adapted to receive wired signals from cartridge 142 (and in some cases to transmit wired signals to cartridge 142).

FIG. 21 illustrates an embodiment of patient support apparatus 20 in which cartridge port 128 is integrated into the Fowler section 44 of support deck 30. In this embodiment, cartridge 142 is releasably coupled to the Fowler section 44 of patient support apparatus 20. When cartridge 142 is inserted into port 128, cartridge receiver 126 (which may also be positioned inside of Fowler section 44 or elsewhere on patient support apparatus 20) is adapted to receive wired signals from cartridge 142 (and in some cases to transmit wired signals to cartridge 142).

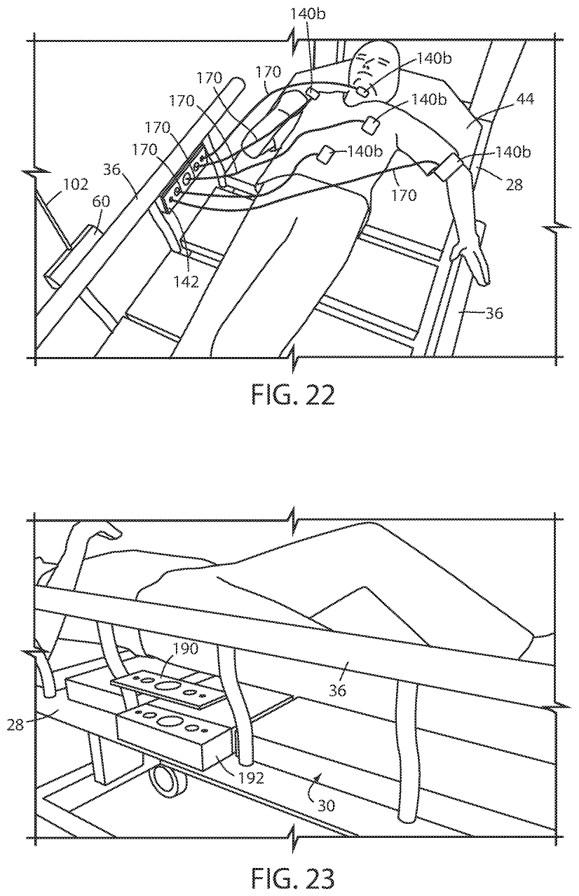

FIG. 22 illustrates another embodiment of patient support apparatus 20 in which cartridge port 128 is integrated into a siderail 36 of patient support apparatus 20. In this embodiment, cartridge 142 is releasably coupled to one of the siderails 36 of patient support apparatus 20. When cartridge 142 is inserted into port 128, cartridge receiver 126 (which may also be positioned inside of the siderail 36 or elsewhere on patient support apparatus 20) is adapted to receive wired signals from cartridge 142 (and in some cases to transmit wired signals to cartridge 142).

FIG. 23 illustrates another embodiment of patient support apparatus 20 in which cartridge port 128 is integrated into the top side of litter frame 28 at a location approximately midway between head end 38 and foot end 40. In this embodiment, cartridge 142 is releasably coupled to litter frame 28 of patient support apparatus 20. When cartridge 142 is inserted into port 128, cartridge receiver 126 (which may also be positioned on top of litter frame 28 or elsewhere on patient support apparatus 20) is adapted to receive wired signals from cartridge 142 (and in some cases to transmit wired signals to cartridge 142).

Cartridge port 128 may also be positioned at other locations on patient support apparatus 20 beyond those illustrated in FIGS. 20-23. Further, in some embodiments, patient support apparatus 20 may be constructed with multiple cartridge ports 128 into which multiple cartridges 142 may be inserted. In such embodiments, a single cartridge 142 may be inserted into different ports 128 at different times, depending upon which port 128 the caregiver deems more convenient at a given moment and/or for different vital sign sensors 140.

In some embodiments, cartridge 142 does not include any UWB transceivers and the association of cartridge 142 (and the vital sign sensors 140b and/or 140c coupled thereto) is carried out in any of the manners discussed above. In such embodiments, controller 132 determines whether to associate, or not associate, a particular cartridge 142 with patient support apparatus 20 without regard to the position of cartridge 142 with respect to space volume 152. In alternative embodiments of cartridge 142, it may include one or more UWB transceivers that are adapted to communicate with UWB transceiver 124 onboard patient support apparatus 20 so that the relative position of cartridge 142 with respect to patient support apparatus 20 may be determined. In these UWB embodiments, controller 132 may utilize the position of cartridge 142 relative to space volume 152 to determine whether to associate a cartridge with patient support apparatus 20 or not. Alternatively, or additionally, controller 132 may also determine the association of cartridge 142 based on the cartridge ID it receives from the cartridge 142 when it is inserted into cartridge ports 128 (i.e. controller 132 associates the cartridge 142 with patient support apparatus 20 when the cartridge is inserted into port 128).

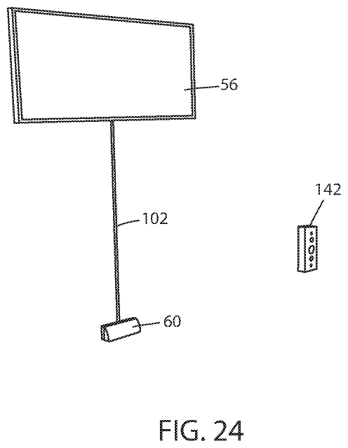

FIG. 24 illustrates an embodiment of cartridge 142 that includes one or more UWB transceivers built into it. In this embodiment, cartridge 142 and/or locator unit 60 are adapted to determine their distance from each other, and if it is less than a threshold, cartridge transmitter 204 is adapted to transmit vital sign readings to locator unit 60 for display on the connected non-UWB display device 56. In this particular embodiment, cartridge 142 need not forward vital sign signals to be displayed on non-UWB display device 56 to patient support apparatus 20, but instead can bypass patient support apparatus 20 and forward such vital sign signals directly to locator unit 60. Cartridge 142 may further be adapted to automatically forward vital sign signals to a UWB display device 156 that comes within a threshold distance of cartridge 142, provided the UWB display device 156 is authorized for such vital sign data display.

The term “associates,” or its variants, as used herein, refers to the identification by controller 132 of which devices (locator units 60, vital sign sensors 140, UWB display device 156, and/or cartridges 142) are intended for use with the patient assigned to patient support apparatus 20 and/or that are intended for use by patient support apparatus 20 at a particular location within a healthcare facility. For locator units 60, patient support apparatus 20 associates itself with only a single locator unit 60 at a given time. For linked locator units 60a, the associated linked locator unit 60a is the linked locator unit 60a that controller 132 will send data to for forwarding to the adjacent communication outlet 64, as well as the linked locator unit 60a that controller 132 will receive data from that originated from communication outlet 64. The associated locator unit 60a is therefore the linked locator unit 60a that patient support apparatus 20 will send the patient's voice signals to (and/or exit detection alerts to) for forwarding to nurse call system 70. It is also the linked locator unit 60a that controller 132 will send television commands to when a patient onboard patient support apparatus 20 activates one or more of the television controls 50l-50r. Similarly, it is the linked locator unit 60a that controller 132 will send light commands to when a patient onboard patient support apparatus 20 activates one or more or the reading or room light controls 50s or 50t. The linked locator unit 60a that patient support apparatus 20 associates itself with is also the linked locator unit 60a that patient support apparatus 20 will receive audio signals from and direct to its onboard speaker(s). Such audio signals may correspond to voice signals from a remotely positioned nurse that are forwarded to the corresponding communication outlet 64 by way of nurse call system 70, or such audio signals may correspond to television audio signals that are routed from television 72 to communication outlet 64 by way of the one or more conductors 68. Controller 132 associates a linked locator unit 60a with patient support apparatus 20 if the linked locator unit 60a is positioned inside of space volume 152 in some embodiments, and in other embodiments, as mentioned, if patient support apparatus 20 is positioned inside of the space volume 152a of that linked locator unit 60a.

Generally speaking, controller 132 is configured to associate with an unlinked locator unit 60b if patient support apparatus 20 moves inside of that unlinked locator unit's space volume 152a. In an alternative embodiment, controller 132 may be configured to associate with an unlinked locator unit 60b if the patient support apparatus 20 moves close enough to the unlinked locator unit 60b such that the locator unit 60b is positioned inside of space volume 152. In still other embodiments, the unlinked locator units 60b that controller 132 associates with may be the nearest unlinked locator unit 60b that is in the same room as, or on the same side of a wall as, patient support apparatus 20.

For vital sign sensors 140, the vital sign sensors 140 that controller 132 associates with are those that are used on the patient who is currently assigned to patient support apparatus 20. In some embodiments, controller 132 presumes that whichever patient is currently occupying patient support apparatus 20 is the patient assigned to patient support apparatus 20. For display devices 156, the display devices 156 that controller 132 associates with are the ones that are intended to display data regarding the patient currently assigned to patient support apparatus 20 and/or data regarding patient support apparatus 20.

For cartridges 142, the cartridge that controller 132 associates with is the cartridge into which one or more vital sign sensors 140b are plugged into and that are being used on the patient assigned to patient support apparatus 20. When 142 cartridge is initially inserted into cartridge port 128, but not yet associated with patient support apparatus 20, cartridge transmitter 204 forwards its cartridge ID 200 to controller 132 (via cartridge receiver 126). Once that ID is received by controller 132, controller 132 associates that cartridge 142 with patient support apparatus 20. Thereafter (i.e. during a post-association period), cartridge transmitter 204 may repetitively send its unique ID 200 with the vital sign data that it sends to patient support apparatus 20. Controller 132 re-checks this cartridge ID 200 to make sure it matches the original cartridge ID that it associated with patient support apparatus 20. To the extent it does, controller 132 continues to process the vital sign data in the manners described herein. To the extent there isn't a match, controller 132 may be configured to stop sending the vital sign data to one or more displays and/or to patient support apparatus server 84, to display an error message on display 52, and/or to send an error message to patient support apparatus server 84. This re-checking process of the cartridge ID 200 is particularly helpful when cartridge 142 is physically removed from port 128 and transmitting vital sign data to patient support apparatus 20 because, in such situations, there is the possibility that another cartridge 142 from another patient support apparatus may be within communication range. By re-checking the cartridge ID after the cartridge has been associated with patient support apparatus 20, controller 132 is able to determine if the wireless data it is receiving is coming from the correct cartridge 142 and, if not, to ignore it.

The task of associating and disassociating a particular patient to a particular patient support apparatus 20 may be carried out locally by controller 132, or it may be carried out remotely by patient support apparatus server 84. Such remote association generally involves patient support apparatus server 84 using information from a conventional Admissions, Discharge, and Transfer (ADT) server or EMR server on network 80 to determine the room location (e.g. room number and/or bed bay ID) of a particular patient, and then matching that room location with the room location of a particular patient support apparatus 20 (which is reported to server 84 by the patient support apparatuses 20 which use locator units 60 to determine their location). In other words, server 84 consults a conventional server on network 80 that correlates specific patients to specific room numbers and/or bay areas, such as an ADT and/or EMR server, and then uses the known room numbers and/or bay areas of specific patient support apparatuses 20 to match a specific patient to a specific patient support apparatus 20.

Local association of a particular patient support apparatus 20 and a particular patient may be carried out in a variety of different manners. One manner involves incorporating a bar code scanner and/or near field sensor into patient support apparatus 20 that is adapted to read the bar code and/or near field patient ID data contained within a conventional patient wristband. When a patient is assigned to a particular patient support apparatus 20, the caregiver scans the patient's wristband using the bar code or near field scanner that is built into the patient support apparatus 20. The patient ID data that is read from the wristband is then forwarded to server 84, which may distribute it to an EMR server and/or to other servers on the network 80. Controller 132 may also be adapted to include this patient ID data in the vital sign readings it reports to server 84 (and/or to another server on network 80) from vital sign sensors 140 so that the servers know which specific patient the readings are from.

In some embodiments of patient support apparatus 20, controller 132 forwards information to patient support apparatus server 84 so that server 84 can determine whether to associate a particular device with a particular patient support apparatus 20. In such embodiments, server 84 sends a message back to patient support apparatus 20 informing it of what devices should be associated with, and what devices should not be associated with, patient support apparatus 20.

In some embodiments, when patient support apparatus 20 and/or server 84 associate a device with a particular patient support apparatus 20, controller 132 and/or server 84 are configured to inform medical personnel (via electronic devices 94) that the device is associated with a particular patient support apparatus 20 and/or with a particular patient assigned to that patient support apparatus 20. In this manner, data from the device can be correlated with a particular patient, room location, and/or patient support apparatus 20. Patient support apparatus 20 may therefore be configured to automatically forward vital sign data (or data from other medical devices) to server 84 after the vital sign sensor 140 (or other medical device) become associated with patient support apparatus 20. Such vital sign data (or other data) may, in turn, be automatically forwarded by server 84 to an EMR server for entry into the corresponding patient's electronic medical record. In this manner, once controller 132 associates a vital sign sensor 140 with the patient support apparatus 20, data from the vital sign sensor can be automatically recorded in that patient's particular electronic medical record without requiring the caregiver to either associate the vital sign sensor with the patient and/or with the patient support apparatus 20 assigned to that patient. In other words, because controller 132 automatically determines that the vital sign sensor 140 (or other medical device) is positioned within space volume 152, it is not necessary for a caregiver to take any manual steps to ensure that data from the vital sign sensor 140 (or other medical device) is forwarded to the proper corresponding patient's electronic medical record because patient support apparatus 20, along with server 84, automatically determine the correct patient associated with that medical device. Further details regarding at least one manner in which this automatic patient determination may be made are found in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which is incorporated herein by reference.

In addition, the aforementioned '777 patent discloses several manners in which a UWB tag may be constructed. Such tags may be attached to vital sign sensors 140 and/or to other medical devices used with patient support apparatus 20. The use of such tags allows controller 132 to automatically determine the position of vital sign sensors 140 and/or other medical devices that don't have their own built-in UWB transceivers, but that instead have a UWB tag attached to them.

In general, the tag attached to a vital sign sensor 140 (or other medical device) includes a UWB transceiver that is able to communicate with each UWB transceiver 124 onboard patient support apparatus 20. This communication enables controller 132 of patient support apparatus 20 to determine the distances between each UWB transceiver 124 and the tag. By knowing these distances, as well as the location of each transceiver 124 on patient support apparatus 20, controller 132 is able to determine the relative position of the tag with respect to patient support apparatus 20, including whether the tag is positioned inside or outside of space volume 152.

It will be understood that a tag may be attached to not only a vital sign sensor 140, but to any device that may be used with a patient and whose location relative to space volume 152 is desirably known. Such medical devices include, but are not limited to, exercise devices, heel care boots, IV stands and/or poles, ventilators, patient monitors (e.g. saturated oxygen (SpO₂) monitors), patient positioning devices (e.g. wedges, turning devices, pumps), ambient sensors (e.g. air temperature, air flow, light, humidity, pressure, altitude, sound/noise), and/or any other types of devices that are used in the treatment, monitoring, and/or rehabilitation of the patient. A built in UWB transceiver may also, or alternatively, be built into any of these medical devices and adapted to communicate with UWB transceiver 124, therefore making the use of a separate tag unnecessary.

In at least one embodiment, patient support apparatus 20 may be configured to detect the position of an exercise device (and/or movement of the exercise device) in any one or more of the manners disclosed in commonly assigned U.S. patent application Ser. No. 63/161,175 filed Mar. 15, 2021, by inventors Krishna Bhimavarapu et al. and entitled EXERCISE DEVICE AND PATIENT SUPPORT APPARATUS, the complete disclosure of which is incorporated herein by reference.

In some embodiments, the transceivers 104, 124, 146, and 160 may operate in the same manner as, and include any of the same functions as, the anchors and pseudo-anchors disclosed in commonly assigned U.S. patent application Ser. No. 63/193,777 filed May 27, 2021, by inventors Thomas Deeds et al. and entitled SYSTEM FOR ASSOCIATING MEDICAL DEVICE DATA, the complete disclosure of which has already been incorporated herein by reference.

In some embodiments, locator units 60 may also be utilized to determine the location of a vital sign sensor 140, a UWB display device 156, and/or another type of medical device, such as is disclosed in commonly assigned U.S. patent application Ser. No. 63/132,514 filed Dec. 31, 2020, by inventors Alexander Bodurka et al. and entitled PATIENT SUPPORT APPARATUS AND MEDICAL DEVICE NETWORKS, and in commonly assigned U.S. patent application Ser. No. 63/154,677 filed Feb. 27, 2021, by inventors Celso Pereira et al. and entitled SYSTEM FOR DETERMINING PATIENT SUPPORT APPARATUS AND MEDICAL DEVICE LOCATION, the complete disclosures of both of which are incorporated herein by reference.

In any of the embodiments disclosed herein, server 84 may be configured to additionally execute a caregiver assistance software application of the type described in the following commonly assigned patent applications: U.S. patent application Ser. No. 62/826,97, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application serial number PCT/US2020/039587 filed Jun. 25, 2020, by inventors Thomas Durlach et al. and entitled CAREGIVER ASSISTANCE SYSTEM, the complete disclosures of which are all incorporated herein by reference. That is, server 84 may be configured to share with one or more electronic devices 94 any of the information shared with the electronic devices disclosed in these aforementioned patent applications. Thus, for example, server 84 may be configured to not only share the location of patient support apparatuses 20 (and any medical devices that may be associated with them) with electronic devices 94, but it may also forward to devices 94 patient vital sign data, patient support apparatus status data (e.g. current siderail position, bed exit status, brake status, height status, scale data, etc.) and/or caregiver rounding data (e.g. when the last rounding was performed for a particular patient, when the next rounds are due, etc.).

In some embodiments, locator units 60a and/or 60b may be configured to send a signal to patient support apparatus 20 (via UWB transceiver 104 and/or via BT transceiver 106) indicating what type of locator unit it is (e.g. whether it is a linked locator unit 60a or an unlinked locator unit 60b). Controller 132 of patient support apparatus 20 uses this information to determine whether or not to forward audio signals from microphone 138 to the locator unit 60 via BT transceiver 122 or UWB transceiver 124. More specifically, if the locator unit is a linked locator unit 60a, controller 132 forwards the audio signals (and linked locator unit 60a then forwards the audio signals to the communications outlet 64). If the locator unit is an unlinked locator unit 60b, controller 132 does not forward the audio signals (but instead forwards them to a nurse call cable port that receives a nurse call cable 66). The audio signals are generated from the patient's voice when he or she is talking to a remotely positioned caregiver via the healthcare facility's nurse call system.

It will also be understood that the number of UWB transceivers on patient support apparatus 20 may vary. In some embodiments, patient support apparatus 20 includes three UWB transceivers 124 positioned at known locations on patient support apparatus 20 that are stored in memory 134. In other embodiments, four UWB transceivers 124 are included. In still other embodiments, fewer than three UWB transceivers 124 are used, such as only a single UWB transceiver or two transceivers. Still other numbers of UWB transceivers 124 may be included.

In some embodiments, linked locator units 60a and/or unlinked locator units 60b may include additional information stored therein that is shared with patient support apparatus 20 when patient support apparatus 20 becomes associated with the locator unit 60. Such additional information may include location information identifying the relative position of the locator unit 60 with respect to one or more other locator units 60 that are positioned nearby. Additionally or alternatively, the locator units 60 may include information regarding the thickness and/or materials of the wall 62 to which it is attached, wherein such information provides an indication to the patient support apparatus 20 of the amount of attenuation that UWB signals will likely experience when traveling through that wall. Additionally or alternatively, the locator units 60 may include information identifying their general location within the healthcare facility (e.g. room 400, bay A of room 302, hallway X, maintenance area Y, radiology department, emergency department, etc.) and/or information identifying a more specific location of the locator units 60 within the healthcare facility (e.g. a set of X,Y,Z coordinates in a frame of reference that includes all, or a portion of, the healthcare facility; a height on the wall 62, a distance from one or more landmarks and/or architectural features within the healthcare facility, and/or other more specific information). In some embodiments, patient support apparatus 20 is adapted to utilize this information to determine its location within the healthcare facility and/or to determine whether it is positioned on the same side of the wall 62 as a particular locator unit 60. In some embodiments, patient support apparatus 20 and/or locator units 60 include any of the same structures, functions, and/or features of any of the patient support apparatuses and/or wall units disclosed in commonly assigned U.S. patent application Ser. No. 63/245,245 filed Sep. 17, 2021, by inventors Kirby Neihouser et al. and entitled SYSTEM FOR LOCATING PATIENT SUPPORT APPARATUSES, the complete disclosure of which has already been incorporated herein by reference.

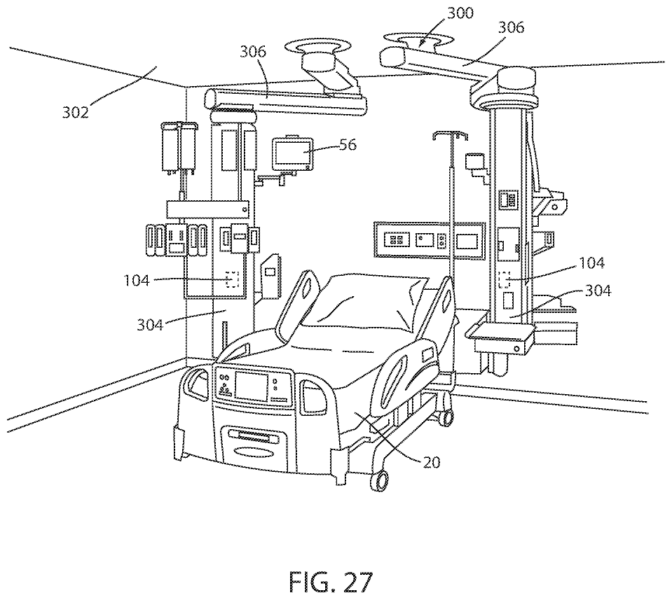

In some embodiments, a linked or unlinked locator unit 60a and/or 60b may be integrated into an overhead boom 300 positioned in a room of a healthcare facility. One example of such a boom 300 is shown in FIG. 27. Boom 300 is attached to a ceiling 302 of the room and includes, in this particular example, two arms 304. Each arm 304 is pivotable about a generally vertical axis, and is attached at its top end to one or more cross bars 306. Each cross bar 306, in turn, is pivotable about another generally vertical axis. In this manner, each arm 304 may be moved to a variety of different positions around the periphery and/or over patient support apparatus 20. In some embodiments, a locator unit 60 (linked or unlinked) is built into overhead boom 300. In such embodiments, the locator unit 60 may include multiple UWB transceivers 104, wherein each arm 304 of the overhead boom 300 includes a UWB transceiver 104. Patient support apparatus 20 is configured to associated with this type of locator unit 60 if at least one of the UWB transceivers 104 is positioned within a predetermined volume of space, such as volume of space 152. In other words, if one of the arms 304 is moved within a close proximity of patient support apparatus 20, but not the other arm 304, patient support apparatus 20 is configured to associate itself with the locator unit 60 integrated into boom 300. Only if both arms 304, and their respective UWB transceivers 104, are positioned outside of the predetermined volume of space will patient support apparatus 20 not associate with, or disassociate itself from, the locator unit 60 built into overhead boom 300.

Although not shown in FIG. 27, the locator unit 60 may include, in additional to the multiple UWB transceiver 104, and and/or all of the components shown in FIG. 5 or 7. When a linked locator unit 60 is built into overhead boom 300, a communication outlet 64 may be integrated into the ceiling 302 at a location where the boom 300 is mounted to the ceiling 302. In some situations, such as that shown in FIG. 27, this ceiling-mounted outlet 64 is concealed from view by the attachment of the boom 300 to the ceiling. In such embodiments, the linked locator unit 60 is configured to act as a communication conduit between an associated patient support apparatus 20 and room devices 72, 74, 76 and/or between an associated patient support apparatus 20 and nurse call system 70. In some embodiments, the predetermined volume of space within which an arm 304 must be positioned in order to associate patient support apparatus 20 with the fixed locator 60 integrated into boom 300 has a different size and/or shape from the predetermined volumes of space 152 that may be used with fixed locators 60 that are mounted to walls 62. In other embodiments, the predetermined volumes of space 152 used with wall-mounted fixed locators 60 and boom-integrated fixed locators 60 may be the same. In some embodiments, such as shown in FIG. 27, a display 56 may be connected to, or integrated into, overhead boom 300, and data may be displayed thereon in any of the manners discussed herein after patient support apparatus 20 is associated with the locator unit 60 built into overhead boom 300.

It will be understood that the communication of vital sign data, or other medical device data, to one or more of the displays 56 and/or 156, as described herein, takes place without routing the data through network 80 and without utilizing network transceiver 96. Instead, this data may be transmitted directly from one UWB transceiver to another, such as from UWB transceiver 146 of vital sign sensor 140a to one of the UWB transceivers 124 of patient support apparatus 20, and from patient support apparatus 20 to locator unit 60 using UWB transceivers 124 and 104. Alternatively, one or more of these devices that transmit, or forward, vital sign data may use Bluetooth communication, or another form of direct communication. By using such direct communication, patient support apparatus 20 reduces the traffic load that would otherwise on network 80.

It will be understood by those skilled in the art that the use of the term “transceiver” throughout this specification is not intended to be limited to devices in which a transmitter and receiver are necessarily within the same housing, or share some circuitry. Instead, the term “transceiver” is used broadly herein to refer to both structures in which circuitry is shared between the transmitter and receiver, and transmitter-receivers in which the transmitter and receiver do not share circuitry and/or a common housing. Thus, the term “transceiver” refers to any device having a transmitter component and a receiver component, regardless of whether the two components are a common entity, separate entities, or have some overlap in their structures.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A patient support apparatus comprising:
a support surface adapted to support a patient;
a first transceiver coupled to a first location on the patient support apparatus;
a second transceiver coupled to a second location on the patient support apparatus;
a third transceiver coupled to a third location on the patient support apparatus; and
a controller adapted to use radio frequency (RF) communication between the first, second, and third transceivers and a fixed locator positioned off-board the patient support apparatus to determine a position of the fixed locator relative to the patient support apparatus, the controller further adapted to send data to be displayed by a display device positioned off-board the patient support apparatus if the fixed locator is positioned inside a predetermined volume of space, and to not send the data to be displayed by the display device if the fixed locator is positioned outside of the predetermined volume of space.

2. The patient support apparatus of claim 1 wherein the first transceiver, the second transceiver, and the third transceiver are all ultra-wideband transceivers, and wherein the patient support apparatus further includes a memory in which the first location, second location, and third location of the first, second, and third transceivers, respectively, is stored.

3. (canceled)

4. The patient support apparatus of claim 2 wherein the controller is further adapted to use the stored locations of the first, second, and third transceivers to determine whether the fixed locator is positioned inside or outside of the predetermined volume of space.

5. The patient support apparatus of claim 1 wherein the controller is further adapted to use RF communication between the first, second, and third transceivers and a vital sign sensor to determine a position of the vital sign sensor relative to the patient support apparatus.

6. The patient support apparatus of claim 5 wherein the controller is further adapted to include readings from the vital sign sensor within the data to be displayed by the display device if the vital sign sensor is positioned inside the predetermined volume of space, and to not include the readings from the vital sign sensor within the data to be displayed by the display device if the vital sign sensor is positioned outside of the predetermined volume of space.

7. The patient support apparatus of claim 6 wherein the display device includes a display and a display controller, and wherein the display controller is adapted to display the data on the display.

8. The patient support apparatus of claim 7 wherein the display device is mounted adjacent a bay of a room within a healthcare facility.

9. The patient support apparatus of claim 6 further comprising a microphone positioned onboard the patient support apparatus, the microphone adapted to convert sounds of the patient's voice to audio signals, and wherein the controller is further adapted to transmit the audio signals to the fixed locator if the fixed locator is positioned inside the predetermined volume of space, and to not transmit the audio signals to the fixed locator if the fixed locator is positioned outside of the predetermined volume of space.

10. The patient support apparatus of claim 9 wherein the display device includes a device controller and a video port adapted to receive a video cable adapted to be coupled to a display, and wherein the device controller is adapted, in response to receiving the data to be displayed by the display device, to send the data to be displayed by the display device to the video port.

11. The patient support apparatus of claim 10 wherein the video port is one of a High-Definition Multimedia Interface (HDMI) connector, a Video Graphics Array (VGA) connector, a DisplayPort (DP) connector, a plurality of Radio Corporation of America (RCA) connectors, or a Digital Visual Interface (DVI) connector.

12. The patient support apparatus of claim 1 further including a vital sign port adapted to receive a plug of a cable from a vital sign sensor adapted to detect a vital sign of the patient, wherein the controller is further adapted to include readings from the vital sign sensor within the data to be displayed by the display device.

13. The patient support apparatus of claim 12 wherein the vital sign port is integrated into a cartridge adapted to be removed from the patient support apparatus while the plug of the cable from the vital sign sensor remains plugged into the vital sign port.

14. The patient support apparatus of claim 13 wherein the cartridge includes an adapter, the cartridge is adapted to be removed from the patient support apparatus with the adapter connected to the cartridge, and the adapter is adapted to be removed from the cartridge while the plug of the cable is coupled to the adapter.

15. The patient support apparatus of claim 14 wherein the cartridge is removably secured to the patient support apparatus via an engineering fit, and the adapter is removably secured to the cartridge by magnetic coupling.

16. The patient support apparatus of claim 1 wherein the controller is adapted to include a room number within the data to be displayed by the display device, the room number corresponding to a room in which the patient support apparatus is currently located.

17. The patient support apparatus of claim 16 further comprising:
a network transceiver adapted to communicate with a server of a local area network of a healthcare facility; and
wherein the controller is adapted to receive a location ID from the fixed locator, to send the location ID to the server using the network transceiver, and to thereafter receive the room number from the server via the network transceiver.

18. The patient support apparatus of claim 1 wherein the fixed locator is adapted to be mounted at a fixed location within a room of a healthcare facility and to transmit a location ID to the patient support apparatus.

19. The patient support apparatus of claim 1 further comprising a local display integrated into the patient support apparatus, wherein the controller is adapted to display a screen on the local display that includes the data to be displayed by the display device.

20. (canceled)

21. The patient support apparatus of claim 1 wherein the predetermined volume of space is defined with respect to the patient support apparatus and moves as the patient support apparatus moves.

22. The patient support apparatus of claim 1 further comprising a user interface adapted to allow a user to control a content of the data to be displayed by the display device.

23-168. (canceled)
